(19) United States
(12) Patent Application Publication
(10) Pub. No.: US 2004/0176391 A1
(43) Pub. Date: Sep. 9, 2004

(54) Aerosolizable pharmaceutical formulation for fungal infection therapy

(75) Inventors: Jeffry G. Weers, Half Moon Bay, CA (US); Thomas E. Tarara, Burlingame, CA (US); Michael A. Eldon, Redwood City, CA (US); Rangachari Narasimhan, Ontario (CA)

Correspondence Address: NEKTAR THERAPEUTICS, 150 INDUSTRIAL ROAD, SAN CARLOS, CA, 94070, US

(73) Assignee: Nektar Therapeutics, San Carlos, CA

(21) Appl. No.: 10/751,342

(22) Filed: Dec. 31, 2003

Related U.S. Application Data
(60) Provisional application No. 60/437,363, filed on Dec. 31, 2002.

(51) Int. Cl. A61L 9/04; A61K 31/496

(52) U.S. Cl. 514/254.07; 424/45

(57) ABSTRACT

A method of treating and/or providing prophylaxis against a pulmonary fungal infection comprises delivering an aerosolized pharmaceutical formulation comprising an antifungal agent to the lungs. The method comprises determining the minimum inhibitory concentration of the antifungal agent for inhibiting pulmonary fungal growth. A sufficient amount of the pharmaceutical formulation is administered to maintain for a period of time a target antifungal agent lung concentration that is greater than the determined minimum inhibitory concentration. In one version, the antifungal agent is amphotericin B.

This application claims the benefit U.S. Provisional Patent Application Serial No. 60/437,363 filed on Dec. 31, 2002, which is incorporated herein by reference in its entirety.

### BACKGROUND

This invention generally relates to a pharmaceutical formulation and to methods for using the pharmaceutical formulation for the treatment and/or prophylaxis of pulmonary fungal infections. The present invention achieves and/or maintains prophylactically effective concentrations of an antifungal agent in the lung with reduced systemic exposure. The antifungal agent may be a polyene antifungal, such as amphotericin B.

Pulmonary fungal infections, such as invasive filamentous pulmonary fungal infection (IFPFI), are major causes of morbidity and mortality in immunocompromised patients. Some diseases, such as AIDS, compromise the immune system. In addition, compromised immune systems are induced when many cancer and transplant patients undergo immunosuppressive therapy. Such immunocompromised patients are all susceptible to pulmonary fungal infections. Severely immunocompromised patients, such as those with prolonged neutropenia or patients requiring 21 or more consecutive days of prednisone at doses of at least 1 mg/kg/day in addition to their other immunosuppressants, are particularly susceptible to the infection. Among immunocompromised patients, overall fungal infection rates range from 0.5 to 28%. Of the autopsied bone marrow transplant patients with idiopathic pneumonia syndrome (IPS) at the Fred Hutchinson Cancer Center, 7.3% had IFPFI. In another study by Vogeser et al, a 4% rate of IFPFI was in 1187 consecutive autopsies performed in European patients dying of any cause during the period from 1993 to 1996. An overwhelming majority of these European patients had been receiving high dose steroid treatment, treatment for a malignancy or had recently received a solid organ transplant or some form of bone marrow transplant.

The most common pulmonary fungal infection in immunocompromised patients is pulmonary aspergillosis. Aspergillosis is a disease caused by Aspergillus fungal species, which invade the body primarily through the lungs. The incidence of Aspergillosis depends on duration and depth of neutropenia and other patient factors such as age, corticosteroid use, prior pulmonary disease, the levels of environmental contamination, the criteria for diagnosis, and how hard the diagnosis is sought. Other filamentous and dimorphic fungi can lead to pulmonary fungal infections. These additional fungi are usually endemic and regional and may include, for example, blastomycosis, disseminated candidiasis, coccidioidomycosis, cryptococcosis, histoplasmosis, mucormycosis, and sporotrichosis. Though typically not affecting the pulmonary system, infections caused by Candida spp., which are usually systemic and most often result from infections via an indwelling device or IV catheter, wound, or a contaminated solid organ transplant, account for 50 to 67% of total fungal infections in immunocompromised patients.

Amphotericin B is the only approved fungicidal compound currently used to treat aspergillosis and is generally delivered intravenously. Amphotericin B is an amphoteric polyene macrolide obtained from a strain of Strptomyces nodosus. Amphotericin B formulated with sodium desoxycholate was the first parental amphotericin B preparation to be marketed. Systemic intravenous therapies are constrained by dose relative toxicities, such as renal toxicity and hepatoxicity, limiting the effectiveness of the treatment and lessening the desirability of the use of amphotericin B prophylactically. Even with the approved therapy, aspergillosis incidence is rising and estimated to kill more than 50% of those infected who receive treatment.

Therefore, it is desirable to be able to provide an effective therapy against fungal infections, particularly pulmonary fungal infections. It is further desirable to be able to safely and effectively treat patients who have developed a pulmonary fungal infection. It is further desirable to be able to provide prophylaxis against fungal infections for patients who will become immunocompromised. It is further desirable to provide a combination of prophylactic therapy and treatment therapy for fungal infections in immunocompromised patients.

### SUMMARY

The present invention satisfies these needs. In one aspect of the invention, an aerosolizable pharmaceutical formulation comprising an antifungal agent is delivered to the lungs of a patient in need of treatment or prophylaxis.

In another aspect of the invention, a method of treating and/or providing prophylaxis against a pulmonary fungal infection comprises determining the minimum inhibitory concentration of an antifungal agent for inhibiting pulmonary fungal growth; and administering an aerosolized pharmaceutical formulation comprising the antifungal agent to the lungs of a patient; wherein a sufficient amount of the pharmaceutical formulation is administered to maintain for at least one week a target antifungal agent lung concentration of at least two times the determined minimum inhibitory concentration.

In another aspect of the invention, a method of treating and/or providing prophylaxis against a pulmonary fungal infection comprises administering an aerosolized pharmaceutical formulation comprising amphotericin B to the lungs of a patient; wherein a sufficient amount of the pharmaceutical formulation is administered to maintain for at least one week a target amphotericin lung concentration of at least 5 μg/g.

In another aspect of the invention, a method of treating or providing prophylaxis against a pulmonary lung infection comprises determining the minimum inhibitory concentration of an antifungal agent for inhibiting pulmonary fungal growth; and administering at least once per week an aerosolized pharmaceutical formulation comprising the antifungal agent to the lungs of a patient; wherein the amount of the pharmaceutical formulation administered is sufficient to maintain for at least three weeks a target antifungal agent lung concentration that is greater than the determined minimum inhibitory concentration.

In another aspect of the invention, a method treating or providing prophylaxis against a pulmonary lung infection comprises administering at least once per week an aerosolized pharmaceutical formulation comprising amphotericin B to the lungs of a patient; wherein the amount of the pharmaceutical formulation administered is sufficient to maintain for at least three weeks a target amphotericin B lung concentration that is greater than the 4 μg/g.

In another aspect of the invention, a method of providing prophylaxis against a pulmonary lung infection comprises determining the minimum inhibitory concentration of an antifungal agent for inhibiting pulmonary fungal growth; administering an aerosolized pharmaceutical formulation comprising the antifungal agent to the lungs of a patient, wherein the amount of the pharmaceutical formulation administered is sufficient to achieve a target antifungal agent lung concentration that is greater than the determined minimum inhibitory concentration; thereafter administering an immunosuppressive agent to the patient for a period of time; and maintaining the target antifungal agent lung concentration throughout the period of time.

In another aspect of the invention, a method of providing prophylaxis against a pulmonary lung infection comprises administering an aerosolized pharmaceutical formulation comprising amphotericin B to the lungs of a patient, wherein the amount of the pharmaceutical formulation administered is sufficient to deliver at least 5 mg of amphotericin B to the lungs per week; thereafter administering an immunosuppressive agent to the patient for a period of time; and administering at least 5 mg of amphotericin B to the lungs per week throughout the period of time.

In another aspect of the invention, a method of treating or providing prophylaxis against a pulmonary lung infection comprises delivering an aerosolized pharmaceutical formulation comprising from 5 mg to 10 mg of amphotericin B to the respiratory tract of a patient once per week for a period of at least two weeks.

In another aspect of the invention, a unit dose receptacle comprises an aerosolizable pharmaceutical formulation for delivering from 5 mg to 10 mg of amphotericin B when aerosolized.

### DRAWINGS

These features, aspects, and advantages of the present invention will become better understood with regard to the following description, appended claims, and accompanying drawings which illustrate exemplary features of the invention. However, it is to be understood that each of the features can be used in the invention in general, not merely in the context of the particular drawings, and the invention includes any combination of these features, where:

FIG. 1 is a graphical representation showing the concentration of amphotericin B at various locations in the body after intratracheal administration and intravenous administration;

FIG. 2 is a graphical representation showing the mean amphotericin B concentration in the lungs of dogs after 14 days of pulmonary administration;

FIG. 3 is a graphical representation of a method of administering a pharmaceutical formulation according to the invention;

FIG. 4 is a graphical representation showing predicted plasma concentration of an antifungal agent administered according to the present invention;

FIG. 5 is a Kapler-Meier Survival Curve showing the effectiveness of the present invention;

FIGS. 6A through 6E are schematic sectional side views showing the operation of a dry powder inhaler that may be used to aerosolize a pharmaceutical formulation according to the invention.

FIG. 7 is a graphical representation showing a plot of flow rate dependence of deposition in an Anderson Cascade Impactor (ACI) for an amphotericin B powder;

FIG. 8 is a graphical representation showing stability of an amphotericin B powder emitted dose efficiency using the Turbospin DPI device at 60 L min⁻¹;

FIG. 9 is a graphical representation showing a plot of stability of an amphotericin B powder aerosol performance using the Turbospin DPI device at 28.3 L min⁻¹;

FIG. 10 is a graphical representation showing a plot of aerosol performance of a pharmaceutical formulation comprising amphotericin B and various phosphatidylcholines; and

FIG. 11 is a graphical representation showing a plot of aerosol performance of a pharmaceutical formulation comprising 70% amphotericin B using various passive DPI devices at 56.6 L min⁻¹.

### DESCRIPTION

The present invention relates to the treatment and/or prophylaxis of fungal infections. Although the process is illustrated in the context of delivering an aerosolizable pharmaceutical formulation comprising an antifungal agent to the lungs, the present invention can be used in other processes and should not be limited to the examples provided herein.

The present invention provides a pharmaceutical formulation and a method of administering the pharmaceutical formulation. The pharmaceutical formulation comprises an antifungal agent for the treatment and/or prophylaxis of a pulmonary fungal infection. Examples of pulmonary fungal infections include aspergillosis, blastomycosis, disseminated candidiasis, coccidioidomycosis, cryptococcosis, histoplasmosis, mucormycosis, sporotrichosis, some infections caused by Candida ssp, and others as known in the art.

The antifungal agent is any agent that has fungistatic or fungicidal properties when present in the lungs of a patient having a pulmonary fungal infection. In one version, the antifungal active agent comprises a polyene antifungal agent, such as amphotericin B. The amphotericin B is particularly preferred in one version of the invention due to its known use and effectiveness. Other polyene antifungal agents include nystatin, hamycin, natamycin, pimaricin, and ambruticin, and pharmaceutically acceptable derivatives and salts thereof. Other suitable antifungal compounds which may be included in an aerosolizable pharmaceutical formulation include acrisocin, aminacrine, anthralin, benanomicin A, benzoic acid, butylparaben, calcium unidecyleneate, candicidin, ciclopirox olamine, cilofungin, clioquinol, clotrimazole, ecaonazole, flucanazole, flucytosine, gentian violet, griseofulvin, haloprogin, ichthammol, iodine, itraconazole, ketoconazole, voriconazole, miconazole, nikkomycin Z, potassium iodide, potassium permanganate, pradimicin A, propylparaben, resorcinol, sodium benzoate, sodium propionate, sulconazole, terconazole, tolnaftate, triacetin, unidecyleneic acid, monocyte-macrophage colony stimulating factor (M-CSF), zinc unidecylenate, and the like. Of these, particularly preferred are candicidin, clotrimazole, econazole, fluconazole, griseofulvin, hamycin, itraconazole, ketoconazole, miconazole, sulconazole, terconazole, voriconazole, and tolnaftate.

In one version, the pharmaceutical formulation is aerosolizabale so that it may be delivered to the lungs of a patient during the patient's inhalation. In this way the antifungal agent in the pharmaceutical formulation is delivered directly to the site of infection. This is advantageous over systemic administration where the agent is delivered to the entire body. Because the antifungal agents often have renal or other toxicity, the amount that may be delivered to the entire body is limited. Therefore, the amount of that may be delivered to the lungs is limited. However, by administering the antifungal agent directly to the lungs, a greater amount may be delivered to the site in need of the therapy while significantly reducing the delivery to other sites in the body.

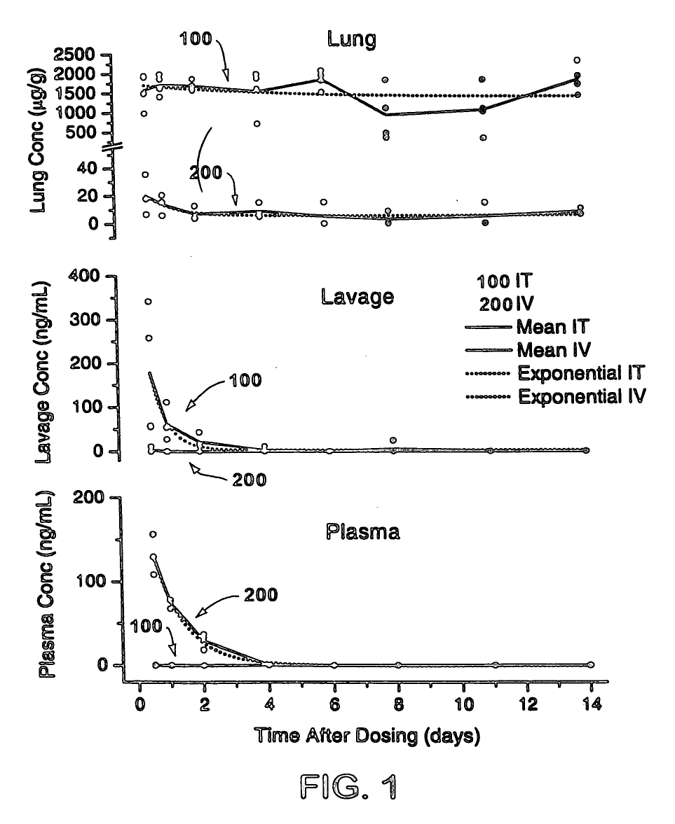

This advantageous therapeutic method is demonstrated by viewing FIG. 1. FIG. 1 shows the concentration of amphotericin B at various locations in the body after delivery of amphotericin B intratracheally 100 and intravenously 200. As can be seen, when administered to the respiratory tract, very little amphotericin B is present in the blood stream thereby significantly reducing the toxic effects of the agent. In contrast, high levels of amphotericin B are present in the blood for up to four days following intravenous administration. As also demonstrated in FIG. 1, the pulmonary concentration of amphotericin B is significantly higher for intratracheal administration 100 than for intravenous administration 200. In the experiment conducted, the lung concentration of amphotericin B is many times greater for intratracheal administration than for intravenous administration while the plasma concentration is less for intratracheal administration. Therefore, by delivering the amphotericin B to the respiratory tract, an effective dose of the pharmaceutical formulation may be delivered to the site of the pulmonary fungal infection, and the undesirable effects of the amphotericin B can be reduced.

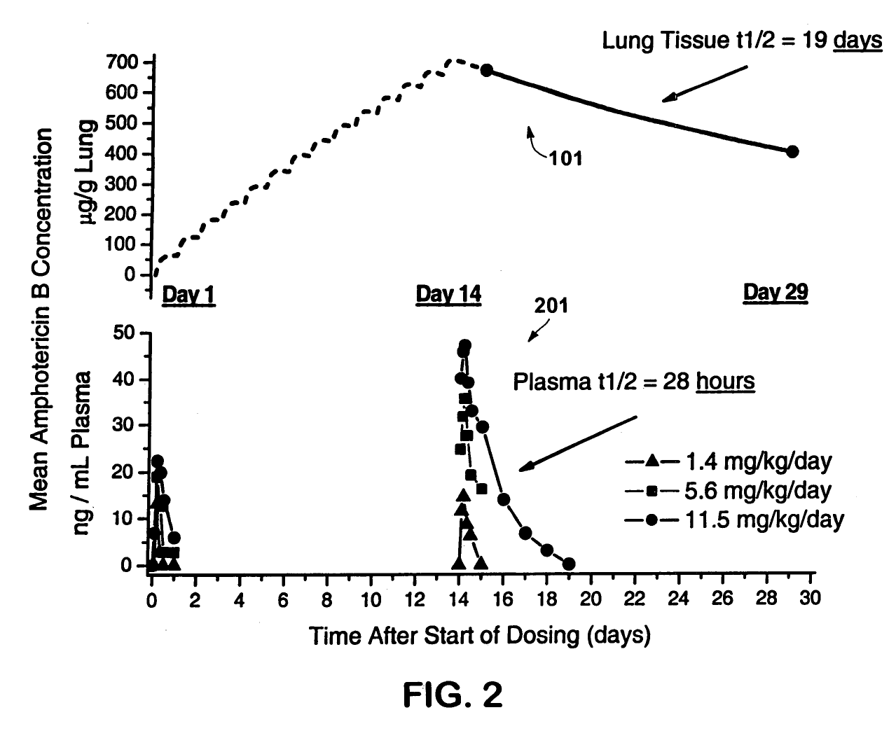

The advantages over intravenous administration are further demonstrated in FIG. 2. FIG. 2 shows the mean amphotericin B concentration in the lungs 101 of dogs following 14 days of pulmonary administration of an aerosolizable pharmaceutical formulation according to the invention. The amphotericin B was delivered in daily doses of 11.5 mg/kg. As can be seen, the amphotericin B resides in the lungs for several days following administration and has a half life of approximately 19 days following administration. In contrast, the intravenous administration 201 is not well retained, having a half life of about 28 hours after administration.

A therapeutic method according to the present invention takes advantage of the lung retention and concentration properties of the pharmaceutical formulation of the present invention to effectively treat a pulmonary fungal infection and/or to provide prophylaxis against a pulmonary fungal infection. In one version, an aerosolizeable pharmaceutical formulation comprising an antifungal agent is administered to the lungs of a patient in a manner that results in an antifungal agent lung concentration greater than a minimum inhibitory concentration (MIC) of the antifungal agent. The MIC is defined as the lowest concentration of active agent that inhibits fungal growth. The MIC may be expressed as a particular concentration value or as a range of concentrations. In one version, a method according to the present invention administers a sufficient amount of the pharmaceutical formulation to achieve a target lung concentration of antifungal that falls within the range of MIC values or is above a particular MIC value. In another version, the target lung concentration of antifungal agent exceeds the MIC range. In another version, the target lung concentration of antifungal agent exceeds the lowest value in an MIC range. In another version, the target lung concentration of antifungal agent is a concentration that exceeds the MIC range and is less than five times the maximum value of the MIC range. The target lung concentration of antifungal agent may be a target lung concentration range. In one version, the target lung concentration range fluctuates above and below a value that is from two to twenty times the midrange value of the MIC range, more preferably that is from three to ten times the midrange value, and most preferably about five times the midrange value. In one version, the antifungal agent concentrations and the MIC determinations are based on the concentrations in the epithelial lining fluid. In another version, the antifungal agent concentrations and the MIC determinations are based on the concentrations in the solid lung tissue. As used herein unless otherwise specified, the MIC value shall be taken to be the particular value when a particular MIC value is determined and shall be taken to be a midrange value when a range of MIC values is determined. MIC determinations may be made according to processes known in the art.

In one version, the pharmaceutical formulation comprising an antifungal agent is administered so that a target lung concentration is maintained over a desired period of time. For example, it has been determined that an administration routine that maintains a target lung concentration of antifungal agent that is at least two times, and more preferably at least three times, the determined MIC value is particularly effective in treating and/or providing prophylaxis against a pulmonary fungal infection. It has been further determined that by maintaining the antifungal lung concentration at the target lung concentration for a period of at least one week, more preferably at least two weeks, and most preferably at least three weeks, a pulmonary fungal infection can be effectively treated in some patients. Additionally or alternatively, by maintaining the antifungal lung concentration at the target concentration for the above periods in an immunocompromised patient, the likelihood of the patient developing a pulmonary fungal infection can be reduced. In many cases, the period of treatment and/or the period of prophylaxis may be extended to be more than one month, more than two months, and sometimes for three months or longer.

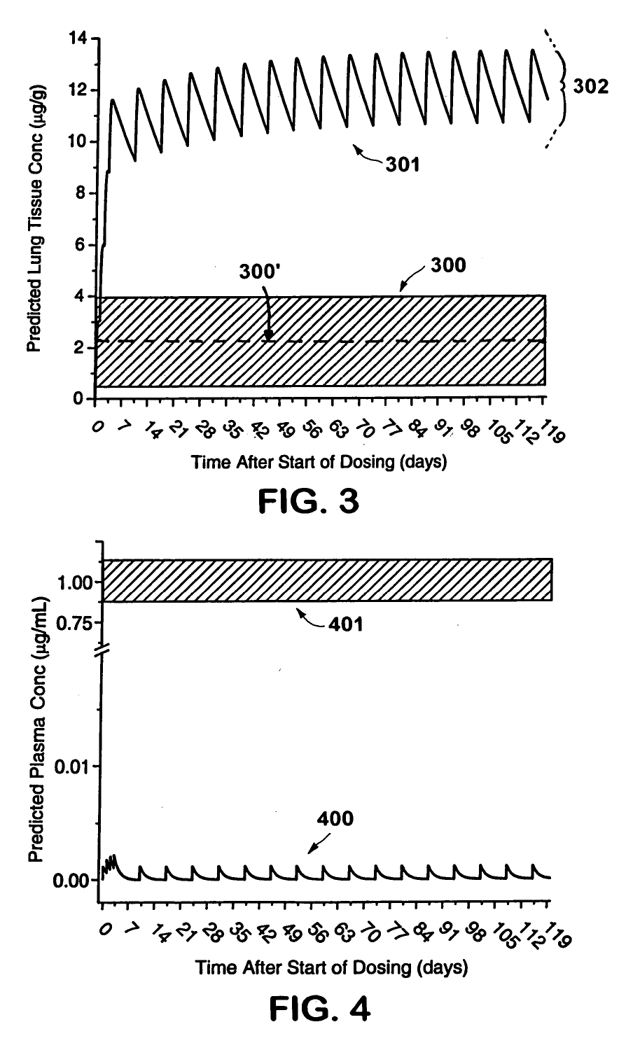

An example of a version of the present invention for administration of aerosolized amphotericin B is shown in FIG. 3. The MIC value for amphotericin B in this version has been determined to be a range of from about 0.5 μg/g to about 4 μg/g, as shown by block 300. The midrange MIC value 300′ is about 2.25 μg/g. The curve 301 shows a predicted lung concentration of amphotericin B according to a particular administration regimen. As can be seen, the concentration of amphotericin B reaches a target lung concentration range 302 that is above the MIC range 300 and is at least two times greater than the midrange MIC value 300′. The target lung concentration range 302 may in this version range from 4 μg/g to 50 μg/g, more preferably from 4.5 μg/g to 20 μg/g. In the specific version shown, the target lung concentration range 302 is a range from 9 μg/g to 15 μg/g, and fluctuates about a concentration value that is about five times the midpoint value 300′ of the MIC range 300.

In the example shown in FIG. 3, the method of administering the amphotericin B takes advantage of the lung retention properties of the pharmaceutical formulation comprising amphotericin B. Once the target lung concentration 302 is reached, the pharmaceutical formulation may be administered once per week in order to maintain the antifungal lung concentration within the target lung concentration. The dosage necessary and the frequency of dosing for maintaining the antifungal agent concentration within the target concentration is dependent upon the formulation and concentration of the antifungal agent within the formulation. In the version shown, the antifungal agent is administered weekly. In this version, the weekly dosage of amphotericin B is from 2 mg to 50 mg, more preferably from 2 mg to 25 mg, more preferably from 4 mg to 20 mg, and most preferably 5 mg to 10 mg. The dose may be administered during a single inhalation or may be administered during several inhalations. The fluctuations of antifungal agent lung concentration can be reduced by administering the pharmaceutical formulation more often or may be increased by administering the pharmaceutical formulation less often. Therefore, the pharmaceutical formulation of the present invention may be administered from three times daily to once a month, more preferably from once daily to once every two weeks, more preferably from once every two days to once a week, and most preferably once per week. In each of the administration regimens, the dosages and frequencies are determined to give a lung concentration that is maintained within a certain target lung concentration.

In one version, the pharmaceutical formulation is administered prophylactically to a patient who is likely to become immunocompromised. For example, a patient who will undergo drug immunosuppressive therapy, such as a patient expecting a bone marrow transplant, can be prophylactically treated with a pharmaceutical formulation comprising an antifungal agent to reduce the likelihood of developing a fungal infection during an immunocompromised period. In this version, the antifungal administration is initiated a sufficient amount of time before the patient is immunocompromised to allow the lung concentration of antifungal agent to reach the target lung concentration on or before the time of immunocompromise. When a dose is administered once weekly, the prophylactic period may vary from 1 to 4 weeks, depending on the active agent, formulation, and dosage. However, in one version of the invention, the prophylactic period is shortened by either providing high doses of active agent during the prophylactic period and/or by more frequently administering the dosages during the prophylactic period. An example of this prophylactic loading is shown in FIG. 3. In this version, additional doses are administered during the first week of therapy. For example, doses may be administered on days 1, 2, 3, and 4 and then on every seventh day thereafter. This early loading allows the target lung concentration to be achieved much sooner. Accordingly, the time for prophylaxis is reduced and a patient may begin his or her immunocompromised period sooner. In the example shown, a patient may become immunocompromised after day seven, sometimes after day four, with a significantly reduced likelihood of developing a pulmonary fungal infection. Additionally or alternatively, the dosage administered during the pre-immunosuppression period may be higher than the dosage administered to maintain the target lung concentration. For example, in one version, the first dose may be at least two times the steady state dosage given once the target concentration has been achieved.

The early loading may also be desireable when treating a patient who has a fungal infection. By early loading, the target lung concentration of antifungal agent in the lungs is achieved sooner than when no early loading is administered. Therefore, the treatment of the pulmonary fungal infection may be more rapidly provided.

In one specific therapeutic method, prophylaxis of pulmonary fungal infections is provided for a patient undergoing immunosuppressive therapy. According to this version, the patient is administered at least 5 mg, more preferably from 5 mg to 10 mg, of aerosolized amphotericin B during the patient's inhalation at least two times per week during an initial period. More preferably, the aerosolized amphotericin B is administered at least three times per week during the initial period. In one version, the initial period may last from one to three weeks. Following the initial period, the patient is administered the same dosage less frequently. For example, the aerosolized amphotericin B may be administered once every two weeks, and more preferably once per week. Following the initial period or near the end of the initial period, the immunosuppressive therapy is initiated. The second period of administration is continued so that the target lung concentration is maintained at least through the period of immunocompromised and longer if needed or if a pulmonary fungal infection develops. Additionally or alternatively, the dosage administered during the first period may be larger than the dosage administered during the second period. For example, during the first period, from 10 mg to 20 mg of amphotericin B may be administered and a lesser amount, such as from 5 mg to 10 mg, is administered during the second period. Optionally, a third dosing period may be provided where the dosage is administered less frequently and/or in a lesser amount than in the second period. The third dosing period may be initiated near the end of an immunocompromised period, such as by being initiated when the immunosuppressive therapy is terminated or reduced in severity.

The maintenance of the antifungal lung concentration within a target lung concentration range according to the present invention is advantageous in its effectiveness in treating and/or providing prophylaxis against fungal infections and is also safer than conventional treatment. FIG. 4 shows the resulting predicted plasma concentration 400 during administration of amphotericin B according to the invention. As can be seen, the amphotericin B is significantly less than the plasma minimum toxicity levels 401, thereby increasing the safety of the administration.

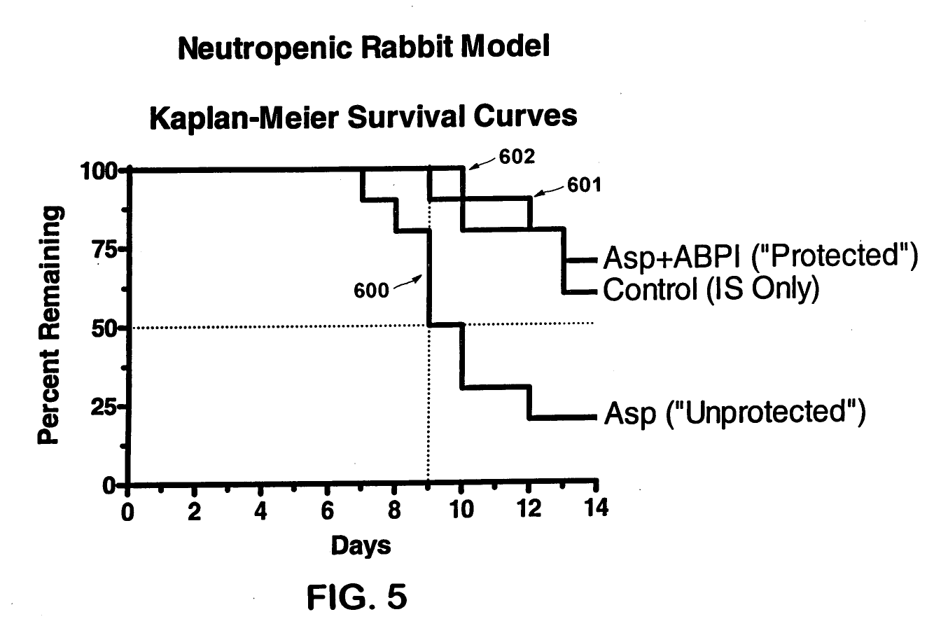

FIG. 5 shows a Kaplan-Meier Survival Curve for neutropenic rabbits. Of the rabbits that were immunosuppressed and were actively exposed to aspergillosis 600, only 50% survived beyond nine days. In contrast, of the rabbits that were immunosuppressed, exposed to aspergillosis, and administered amphotericin B according to the invention 601, 100% survived beyond nine days. Curve 602 shows a control group of rabbits that were immunosuppressed only. In the longer term, less than 25% of the untreated exposed rabbits 600 survived beyond 14 days whereas about 70% of the treated and exposed rabbits 602 survived beyond 14 days.

The pharmaceutical formulation according to the invention may comprise an antifungal agent and optionally one or more additives. For example, the pharmaceutical formulation may comprise neat particles of antifungal agent, may comprise neat particles of antifungal agent together with other particles, and/or may comprise particles comprising antifungal agent and one or more additives. The pharmaceutical formulation of the present invention allow for the delivery of an antifungal agent with improved or enhanced bioavailability, delivery efficiency, chemical stability, physical stability, and/or producibility. In one version, the pharmaceutical formulation comprises an antifungal agent, which may be in amorphous or crystalline form, at least partially incorporated in a matrix material. The matrix material is selected to provide desired characteristics, such as aerosol dispersibility or improved suspension within a liquid medium. The pharmaceutical formulation of the present invention may be formed for extended release or for immediate release.

When the antifungal agent is insoluble, such as by having a solubility in water of less than 1.0 mg/ml, then the pharmaceutical formulation comprises an antifungal agent particle that is in a matrix material. Accordingly, when the antifungal agent is amphotericin B, then the pharmaceutical formulation may comprise amphotericin B particles in a matrix material. It has been discovered that it is advantageous to use small diameter insoluble antifungal agent particles. In particular for an aerosolizable pharmaceutical formulation, it has been determined to be desirable to use antifungal agent particles that are less than 3 μm in diameter. Accordingly, in one version, the pharmaceutical formulation of the present invention is produced using insoluble antifungal agent particles, at least 20% of which have a diameter less than 3 μm, and more preferably at least 50% of which have a diameter less than 3 μm. In a preferred version, at least 90% of the mass of particles of active agent used to make the pharmaceutical formulation are less than 3.0 μm in diameter, more preferably at least 95% of the mass of particles of active agent used to make the pharmaceutical formulation are less than 3.0 μm in diameter. Alternatively or additionally, at least 50% of the mass of particles of active agent used to make the pharmaceutical formulation are between 0.5 μm and 3.0 μm in diameter, and more preferably between 1.0 μm and 3.0 μm. In another version, it is desirable for the antifungal agent particles to be less than 2.5 μm, and more preferably less than 2.0 μm. Accordingly, in this version, the pharmaceutical formulation of the present invention is produced using antifungal agent particles, most of which have a diameter less than 2.5 μm, and more preferably less than 2.0 μm. In one version, at least 90% of the mass of particles of active agent used to make the pharmaceutical formulation are less than 2.5 μm in diameter, more preferably at least 95% of the mass of particles of active agent used to make the pharmaceutical formulation are less than 2.5 μm in diameter. Alternatively or additionally, at least 50% of the mass of particles of active agent used to make the pharmaceutical formulation are between 0.5 μm and 2.5 μm in diameter, and more preferably between 1.0 μm and 2.5 μm. The antifungal agent particle may be in crystalline form.

In many instances, the insoluble antifungal agent in bulk form has a particle size greater than 3.0 μm, and in many cases greater than 10 μm. Accordingly, in one version of the invention, the bulk insoluble antifungal agent is subjected to a size reduction process to reduce the particle size to below 3 microns prior to incorporating the antifungal agent particles in the matrix material. Suitable size reduction processes are known in the art and include supercritical fluid processing methods such as disclosed in WO 95/01221, WO 96/00610, and WO 98/36825, cryogenic milling, wet milling, ultrasound, high pressure homogenization, microfluidization, crystallization processes, and in processes disclosed in U.S. Pat. No. 5,858,410, all of which are incorporated herein by reference in their entireties. Once the desired particle size of the insoluble antifungal agent has been achieved, the resulting antifungal agent particles are collected and then incorporated into a matrix material.

It has been unexpectedly discovered that it is particularly advantageous for the particle size of the insoluble antifungal agent particles to be below 3.0 μm, preferably below 2.5 μm, and most preferably below about 2.0 μm, in order to provide highly dispersible, homogenous compositions of active agent incorporated into the matrix material. It has been discovered that if the insoluble antifungal agent particle size is greater than about 3.0 microns, a heterogeneous composition results comprising active agent incorporated in the matrix material and particles comprising active agent without any matrix material. These heterogeneous compositions often exhibit poor powder flow and dispersibility. Accordingly, a preferred embodiment is directed to homogeneous compositions of insoluble antifungal agent incorporated in a matrix material without any unincorporated active agents particles. However, in some cases, such heterogeneous compositions may be desirable in order to provide a desired pharmacokinetic profile of the active agent to be administered, and in these cases, a large insoluble antifungal agent particle may be used.

In one version, the antifungal agent is incorporated in a matrix that forms a discrete particulate, and the pharmaceutical formulation comprises a plurality of the discrete particulates. The particulates may be sized so that they are effectively administered and/or so that they are highly bioavailable. For example, for an aerosolizable pharmaceutical formulation, the particulates are of a size that allows the particulates to be aerosolized and delivered to a user's respiratory tract during the user's inhalation. Accordingly, in one version, the pharmaceutical formulation comprises particulates having a mass median diameter less than 20 μm, more preferably less than 10 μm, and more preferably less than 5 μm.

The matrix material may comprise a hydrophobic or a partially hydrophobic material. For example, the matrix material may comprise a lipid, such as a phospholipid, and/or a hydrophobic amino acids, such as leucine and tri-leucine. Examples of phospholipid matrices are described in PCT Publications WO 99/16419, WO 99/16420, WO 99/16422, WO 01/85136 and WO 01/85137 and in U.S. Pat. Nos. 5,874,064; 5,855,913; 5,985,309; and 6,503,480, and in copending and co-owned U.S. Patent Application entitled “Pharmaceutical Formulation with an Insoluble Active Agent” to Weers et al., filed on Dec. 31, 2003, Nektar Docket No. 0101.00, all of which are incorporated herein by reference in their entireties. Examples of hydrophobic amino acid matrices are described in U.S. Pat. Nos. 6,372,258 and 6,358,530, and in U.S. patent application Ser. No. 10/032,239 filed on Dec. 21, 2001, all of which are incorporated herein by reference in their entireties.

The pharmaceutical formulation may be advantageously produced using a spray drying process. In one version, the antifungal agent and the matrix material are added to an aqueous feedstock to form a feedstock solution, suspension, or emulsion. The feedstock is then spray dried to produce dried particulates comprising the matrix material and the antifungal agent. Suitable spray drying processes are known in the art, for example as disclosed in PCT WO 99/16419 and U.S. Pat. Nos. 6,077,543, 6,051,256, 6,001,336, 5,985,248, and 5, 976,574, all of which are incorporated herein by reference in their entireties.

In one version, the pharmaceutical formulation comprises a saturated phospholipid, such as one or more phosphatidylcholines. Preferred acyl chain lengths are 16:0 and 18:0 (i.e. palmitoyl and stearoyl). The phospholipid content may be determined by the active agent activity, the mode of delivery, and other factors. In general, the phospholipid content is in the range from about 5% to up to 99.9% w/w, preferably 20% w/w-80% w/w. Thus, antifungal agent loading can vary between about 0.1% and 95% w/w, preferably 20-80% w/w.

Phospholipids from both natural and synthetic sources are compatible with the present invention and may be used in varying concentrations to form the structural matrix. Generally compatible phospholipids comprise those that have a gel to liquid crystal phase transition greater than about 40° C. Preferably the incorporated phospholipids are relatively long chain (i.e. C₁₆-C₂₂) saturated lipids and more preferably comprise saturated phospholipids, as discussed above. Exemplary phospholipids useful in the disclosed stabilized preparations comprise, phosphoglycerides such as dipalmitoylphosphatidylcholine, disteroylphosphatidylcholine, diarachidoylphosphatidylcholine dibehenoylphosphatidylcholine, diphosphatidyl glycerol, short-chain phosphatidylcholines, long-chain saturated phosphatidylethanolamines, long-chain saturated phosphatidylserines, long-chain saturated phosphatidylglycerols, long-chain saturated phosphatidylinositols.

When phospholipids are utilized as the matrix material, the pharmaceutical formulation may also comprise a polyvalent cation, as disclosed in WO PCT 01/85136 and WO 01/85137, hereby incorporated in their entirety by reference. Suitable polyvalent cations are preferably a divalent cation including calcium, magnesium, zinc, iron, and the like. The polyvalent cation may be present in an amount effective to increase the Tm of the phospholipid such that the particulate composition exhibits a Tm which is greater than its storage temperature Ts by at least 20° C., preferably at least 40° C. The molar ratio of polyvalent cation to phospholipid should be at least 0.05, preferably 0.05-2.0, and most preferably 0.25-1.0. A molar ratio of polyvalent cation:phospholipid of about 0.50 is particularly preferred. Calcium is the particularly preferred polyvalent cation and is provided as calcium chloride.

In addition to the phospholipid, a co-surfactant or combinations of surfactants, including the use of one or more in the liquid phase and one or more associated with the particulate compositions are contemplated as being within the scope of the invention. By “associated with or comprise” it is meant that the particulate compositions may incorporate, adsorb, absorb, be coated with or be formed by the surfactant. Surfactants include fluorinated and nonfluorinated compounds and are selected from the group consisting of saturated and unsaturated lipids, nonionic detergents, nonionic block copolymers, ionic surfactants and combinations thereof. In those embodiments comprising stabilized dispersions, such nonfluorinated surfactants will preferably be relatively insoluble in the suspension medium. It should be emphasized that, in addition to the aforementioned surfactants, suitable fluorinated surfactants are compatible with the teachings herein and may be used to provide the desired preparations.

Compatible nonionic detergents suitable as co-surfactants comprise: sorbitan esters including sorbitan trioleate (Span™ 85), sorbitan sesquioleate, sorbitan monooleate, sorbitan monolaurate, polyoxyethylene (20) sorbitan monolaurate, and polyoxyethylene (20) sorbitan monooleate, oleyl polyoxyethylene (2) ether, stearyl polyoxyethylene (2) ether, lauryl polyoxyethylene (4) ether, glycerol esters, and sucrose esters. Other suitable nonionic detergents can be easily identified using McCutcheon's Emulsifiers and Detergents (McPublishing Co., Glen Rock, N.J.) which is incorporated herein in its entirety. Preferred block copolymers include diblock and triblock copolymers of polyoxyethylene and polyoxypropylene, including poloxamer 188 (Pluronic™ F-68), poloxamer 407 (Pluronic™ F-127), and poloxamer 338. Ionic surfactants such as sodium sulfosuccinate, and fatty acid soaps may also be utilized.

Other lipids including glycolipids, ganglioside GM1, sphingomyelin, phosphatidic acid, cardiolipin; lipids bearing polymer chains such as polyethylene glycol, chitin, hyaluronic acid, or polyvinylpyrrolidone; lipids bearing sulfonated mono-, di-, and polysaccharides; fatty acids such as palmitic acid, stearic acid, and oleic acid; cholesterol, cholesterol esters, and cholesterol hemisuccinate may also be used in accordance with the teachings of this invention.

It will further be appreciated that the pharmaceutical formulation according to the invention may, if desired, contain a combination of two or more active ingredients, such as two or more antifungal agents or an antifungal agent and another active agent. The agents may be provided in combination in a single species of particulate composition or individually in separate species of particulate compositions. For example, two or more active agents may be incorporated in a single feed stock preparation and spray dried to provide a single particulate composition species comprising a plurality of active agents. Conversely, the individual actives could be added to separate stocks and spray dried separately to provide a plurality of particulate composition species with different compositions. These individual species could be added to the suspension medium or dry powder dispensing compartment in any desired proportion and placed in the aerosol delivery system as described below. Further, the pharmaceutical formulation may be combined with one or more other active or bioactive agents to provide the desired dispersion stability or powder dispersibility.

The pharmaceutical formulation of the present invention may also include a biocompatible, preferably biodegradable polymer, copolymer, or blend or other combination thereof. In this respect useful polymers comprise polylactides, polylactide-glycolides, cyclodextrins, polyacrylates, methylcellulose, carboxymethylcellulose, polyvinyl alcohols, polyanhydrides, polylactams, polyvinyl pyrrolidones, polysaccharides (dextrans, starches, chitin, chitosan, etc.), hyaluronic acid, proteins, (albumin, collagen, gelatin, etc.). Examples of polymeric resins that would be useful for the preparation of perforated ink microparticles include: styrene-butadiene, styrene-isoprene, styrene-acrylonitrile, ethylene-vinyl acetate, ethylene-acrylate, ethylene-acrylic acid, ethylene-methylacrylatate, ethylene-ethyl acrylate, vinyl-methyl methacrylate, acrylic acid-methyl methacrylate, and vinyl chloride-vinyl acetate. Those skilled in the art will appreciate that, by selecting the appropriate polymers, the delivery efficiency of the particulate compositions and/or the stability of the dispersions may be tailored to optimize the effectiveness of the active or agent.

Besides the aforementioned polymer materials and surfactants, it may be desirable to add other excipients to a particulate composition to improve particle rigidity, production yield, emitted dose and deposition, shelf-life and patient acceptance. Such optional excipients include, but are not limited to: coloring agents, taste masking agents, buffers, hygroscopic agents, antioxidants, and chemical stabilizers. Further, various excipients may be incorporated in, or added to, the particulate matrix to provide structure and form to the particulate compositions (i.e. microspheres such as latex particles). In this regard it will be appreciated that the rigidifying components can be removed using a post-production technique such as selective solvent extraction.

Other excipients may include, but are not limited to, carbohydrates including monosaccharides, disaccharides and polysaccharides. For example, monosaccharides such as dextrose (anhydrous and monohydrate), galactose, mannitol, D-mannose, sorbitol, sorbose and the like; disaccharides such as lactose, maltose, sucrose, trehalose, and the like; trisaccharides such as raffinose and the like; and other carbohydrates such as starches (hydroxyethylstarch), cyclodextrins and maltodextrins. Other excipients suitable for use with the present invention, including amino acids, are known in the art such as those disclosed in WO 95/31479, WO 96/32096, and WO 96/32149. Mixtures of carbohydrates and amino acids are further held to be within the scope of the present invention. The inclusion of both inorganic (e.g. sodium chloride, etc.), organic acids and their salts (e.g. carboxylic acids and their salts such as sodium citrate, sodium ascorbate, magnesium gluconate, sodium gluconate, tromethamine hydrochloride, etc.) and buffers is also contemplated. The inclusion of salts and organic solids such as ammonium carbonate, ammonium acetate, ammonium chloride or camphor are also contemplated.

Yet another version of the pharmaceutical formulation include particulate compositions that may comprise, or may be coated with, charged species that prolong residence time at the point of contact or enhance penetration through mucosae. For example, anionic charges are known to favor mucoadhesion while cationic charges may be used to associate the formed microparticulate with negatively charged bioactive agents such as genetic material. The charges may be imparted through the association or incorporation of polyanionic or polycationic materials such as polyacrylic acids, polylysine, polylactic acid and chitosan.

Whatever components are selected, the first step in particulate production typically comprises feedstock preparation. The concentration of the antifungal agent used is dependent on the amount of agent required in the final powder and the performance of the delivery device employed (e.g., the fine particle dose for a MDI or DPI). As needed, cosurfactants such as poloxamer 188 or span 80 may be dispersed into this annex solution. Additionally, excipients such as sugars and starches can also be added.

Optionally, a polyvalent cation-containing oil-in-water emulsion may then be formed in a separate vessel. The oil employed is preferably a fluorocarbon (e.g., perfluorooctyl bromide, perfluorooctyl ethane, perfluorodecalin) which is emulsified with a phospholipid. For example, polyvalent cation and phospholipid may be homogenized in hot distilled water (e.g., 60° C.) using a suitable high shear mechanical mixer (e.g., Ultra-Turrax model T-25 mixer) at 8000 rpm for 2 to 5 minutes. Typically 5 to 25 g of fluorocarbon is added dropwise to the dispersed surfactant solution while mixing. The resulting polyvalent cation-containing perfluorocarbon in water emulsion is then processed using a high pressure homogenizer to reduce the particle size. Typically the emulsion is processed at 12,000 to 18,000 psi, 5 discrete passes.

The antifungal agent suspension or solution and perfluorocarbon emulsion are then combined and fed into the spray dryer. Operating conditions such as inlet and outlet temperature, feed rate, atomization pressure, flow rate of the drying air, and nozzle configuration can be adjusted in accordance with the manufacturer's guidelines in order to produce the required particle size, and production yield of the resulting dry particles. Exemplary settings are as follows: an air inlet temperature between 60° C. and 170° C.; an air outlet between 40° C. to 120° C.; a feed rate between 3 ml to about 15 ml per minute; and an aspiration air flow of 300 L/min. and an atomization air flow rate between 25 to 50 L/min. The selection of appropriate apparatus and processing conditions are well within the purview of a skilled artisan in view of the teachings herein and may be accomplished without undue experimentation. In any event, the use of these and substantially equivalent methods provide for the formation of aerodynamically light microparticles with particle diameters appropriate for aerosol deposition into the lung.

The pharmaceutical formulation may be formulated to comprise particulates that may be used in the form of dry powders or in the form of stabilized dispersions comprising a non-aqueous phase. Accordingly, the dispersions or powders of the present invention may be used in conjunction with metered dose inhalers (MDIs), as described in PCT Publication WO99/16422, with dry powder inhalers (DPIs), as described in PCT Publication WO99/16419, nebulizers, as described in PCT Publication WO99/16420, and/or in liquid dose instillation (LDI) techniques, as described in PCT Publication WO99/16421, to provide for effective drug delivery.

In one version, the pharmaceutical formulation may be delivered to the lungs of a user in the form of a dry powder. Accordingly, the pharmaceutical formulation comprises a dry powder that may be effectively delivered to the deep lungs or to another target site. The pharmaceutical formulation according to this version of the invention is in the form of a dry powder which is composed of particles having a particle size selected to permit penetration into the alveoli of the lungs. Ideally for this delivery, the mass median aerodynamic diameter of the particles is less than 5 μm, and preferably less than 3 μm, and most preferably between 1 μm and 3 μm. The mass median diameter of the particles may be less than 20 μm, more preferably less than 10 μm, more preferably less than 6 μm, and most preferably from 2 μm to 4 μm. The delivered dose efficiency (DDE) of these powders may be greater than 30%, more preferably greater than 40%, more preferably greater than 50%, more preferably greater than 60%, and most preferably greater than 70%. These dry powders have a moisture content less than about 15% by weight, more preferably less than about 10% by weight, and most preferably less than about 5% by weight. Such powders are described in WO 95/24183, WO 96/32149, WO 99/16419, WO 99/16420, and WO 99/16422, all of which are all incorporated herein by reference in their entireties.

“Mass median diameter” or “MMD” is a measure of median particle size, since the powders of the invention are generally polydisperse (i.e., consist of a range of particle sizes). MMD values as reported herein are determined by centrifugal sedimentation and/or by laser defraction, although any number of commonly employed techniques can be used for measuring mean particle size.

“Mass median diameter” or “MMAD” is a measure of mean particle size, since the powders of the invention are generally polydisperse (i.e., consist of a range of particle sizes). “Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, generally in air, as the particle. The aerodynamic diameter encompasses particle shape, density and physical size of a particle. As used herein, MMAD refers to the midpoint or median of the aerodynamic particle size distribution of an aerosolized powder determined by cascade impaction.

In one version, the pharmaceutical formulation comprises an antifungal agent incorporated into a phospholipid matrix. The pharmaceutical formulation may comprise phospholipid matrices that incorporate the active agent and that are in the form of particulates that are hollow and/or porous microstructures, as described in the aforementioned in WO 99/16419, WO 99/16420, WO 99/16422, WO 01/85136 and WO 01/85137. The hollow and/or porous microstructures are particularly useful in delivering the active agent to the lungs because the density, size, and aerodynamic qualities of the hollow and/or porous microstructures are ideal for transport into the deep lungs during a user's inhalation. In addition, the phospholipid-based hollow and/or porous microstructures reduce the attraction forces between particles, making the pharmaceutical formulation easier to deagglomerate during aerosolization and improving the flow properfies of the pharmaceutical formulation making it easier to process. The hollow and/or porous microstructures may exhibit, define or comprise voids, pores, defects, hollows, spaces, interstitial spaces, apertures, perforations or holes, and may be spherical, collapsed, deformed or fractured particulates.

The hollow and/or porous microstructures may be formed by spray drying, as disclosed in WO 99/16419. The spray drying process results in the formation of a pharmaceutical formulation comprisingparticulates having a relatively thin porous wall defining a large internal void. The spray drying process is also often advantageous over other processes in that the particles formed are less likely to rupture during processing or during deagglomeration. The preparation to be spray dried or feedstock can be any solution, course suspension, slurry, colloidal dispersion, or paste that may be atomized using the selected spray drying apparatus. For the case of insoluble antifungal agents, the feedstock may comprise a suspension as described above. Alternatively, a dilute solution and/or one or more solvents may be utilized in the feedstock. In preferred embodiments the feed stock will comprise a colloidal system such as an emulsion, reverse emulsion, microemulsion, multiple emulsion, particulate dispersion, or slurry. Typically the feed is sprayed into a current of warm filtered air that evaporates the solvent and conveys the dried product to a collector. The spent air is then exhausted with the solvent. Commercial spray dryers manufactured by Buchi Ltd. or Niro Corp. may be modified for use to produce the pharmaceutical formulation. Examples of spray drying methods and systems suitable for making the dry powders of the present invention are disclosed in U.S. Pat. Nos. 6,077,543, 6,051,256, 6,001,336, 5,985,248, and 5,976,574, all of which are incorporated herein by reference in their entireties.

In some instances dispersion stability and dispersibility of the spray dried pharmaceutical formulation can be improved by using a blowing agent, as described in the aforementioned WO 99/16419. This process forms an emulsion, optionally stabilized by an incorporated surfactant, typically comprising submicron droplets of water immiscible blowing agent dispersed in an aqueous continuous phase. The blowing agent may be a fluorinated compound (e.g. perfluorohexane, perfluorooctyl bromide, perfluorooctyl ethane, perfluorodecalin, perfluorobutyl ethane) which vaporizes during the spray-drying process, leaving behind generally hollow, porous aerodynamically light microspheres. Other suitable liquid blowing agents include nonfluorinated oils, chloroform, Freons, ethyl acetate, alcohols, hydrocarbons, nitrogen, and carbon dioxide gases.

Although the particulate compositions are preferably formed using a blowing agent as described above, it will be appreciated that, in some instances, no additional blowing agent is required and an aqueous dispersion of the medicament and/or excipients and surfactant(s) are spray dried directly. In such cases, the pharmaceutical formulation may possess special physicochemical properties (e.g., high crystallinity, elevated melting temperature, surface activity, etc.) that makes it particularly suitable for use in such techniques.

In one version, the pharmaceutical formulation is formed by spray drying a feedstock. The first step in the particulate production typically comprises feedstock preparation. If the phospholipid based particulate is intended to act as a carrier for an antifungal agent, the selected active agent is introduced into a liquid, such as water, to produce a concentrated solution or suspension. The polyvalent cation may be added to the active agent solution or may be added to the phospholipid emulsion as discussed below. The active agent may also be dispersed directly in the emulsion. Alternatively, the active agent may be incorporated in the form of a solid particulate dispersion. The concentration of the active agent used is dependent on the amount of agent required in the final powder and the performance of the delivery device employed. In one version, a polyvalent cation-containing oil-in-water emulsion is then formed in a separate vessel. The oil employed is preferably a fluorocarbon (e.g., distearoyl phosphatidylcholine, perfluorooctyl bromide, perfluorooctyl ethane, perfluorodecalin) which is emulsified with a phospholipid. For example, polyvalent cation and phospholipid may be homogenized in hot distilled water (e.g., 60.degree. C.) using a suitable high shear mechanical mixer (e.g., Ultra-Turrax model T-25 mixer) at 8000 rpm for 2 to 5 minutes. Typically 5 to 25 g of fluorocarbon is added dropwise to the dispersed surfactant solution while mixing. The resulting polyvalent cation-containing perfluorocarbon in water emulsion is then processed using a high pressure homogenizer to reduce the particle size. Typically the emulsion is processed at 12,000 to 18,000 psi, 5 discrete passes and kept at 50 to 80.degree. C. The active agent and perfluorocarbon emulsion are then fed into the spray dryer.

Operating conditions such as inlet and outlet temperature, feed rate, atomization pressure, flow rate of the drying air, and nozzle configuration can be adjusted in order to produce the required particle size, and production yield of the resulting dry particles. Exemplary settings are as follows: an air inlet temperature between 60.degree. C. and 170.degree. C.; an air outlet between 40.degree. C. to 120.degree. C.; a feed rate between 3 ml to about 15 ml per minute; and an aspiration air flow of 300 L/min. and an atomization air flow rate between 25 to 50 L/min. The use of the described method provides for the formation of hollow and/or porous microstructures that are aerodynamically light microparticles with particle diameters appropriate for aerosol deposition into the lung, as discussed above.

Particulate compositions useful in the present invention may alternatively be formed by lyophilization. Lyophilization is a freeze-drying process in which water is sublimed from the composition after it is frozen. The particular advantage associated with the lyophilization process is that biologicals and pharmaceuticals that are relatively unstable in an aqueous solution can be dried without elevated temperatures, and then stored in a dry state where there are few stability problems. With respect to the instant invention such techniques are particularly compatible with the incorporation of peptides, proteins, genetic material and other natural and synthetic macromolecules in particulate compositions without compromising physiological activity. The lyophilized cake containing a fine foam-like structure can be micronized using techniques known in the art to provide the desired sized particles.

In one version, the pharmaceutical formulation is composed of hollow and/or porous microstructures having a bulk density less than 0.5 g/cm³, more preferably less than 0.3 g/cm³, more preferably less than 0.2 g/cm³, and sometimes less 0.1 g/cm³. By providing particles with very low bulk density, the minimum powder mass that can be filled into a unit dose container is reduced, which eliminates the need for carrier particles. That is, the relatively low density of the powders of the present invention provides for the reproducible administration of relatively low dose pharmaceutical compounds. Moreover, the elimination of carrier particles will potentially minimize throat deposition and any “gag” effect, since large lactose particles will impact the throat and upper airways due to their size.

The powder pharmaceutical formulation may be administered using an aerosolization device. The aerosolization device may be a nebulizer, a metered does inhaler, a liquid dose instillation device, or a dry powder inhaler. The powder pharmaceutical formulation may be delivered by a nebulizer as described in WO 99/16420, by a metered dose inhaler as described in WO 99/16422, by a liquid dose instillation apparatus as described in WO 99/16421, and by a dry powder inhaler as described in U.S. patent application Ser. No. 09/888,311 filed on Jun. 22, 2001, in WO 02/83220, in U.S. Pat. No. 6,546,929, and in U.S. patent application Ser. No. 10/616,448 filed on Jul. 8, 2003, all of these patents and patent applications being incorporated herein by reference in their entireties.

In one version, the pharmaceutical formulation is in dry powder form and is contained within a unit dose receptacle which may be inserted into or near the aerosolization apparatus to aerosolize the unit dose of the pharmaceutical formulation. This version is useful in that the dry powder form may be stably stored in its unit dose receptacle for a long period of time. In addition, this version is convenient in that no refrigeration or external power source is required for aerosolization.

In some instances, it is desirable to deliver a unit dose, such as doses of 5 mg or greater of active agent to the lung in a single inhalation. The above described phospholipid hollow and/or porous dry powder particulates allow for doses of 5 mg or greater, often greater than 10 mg, and sometimes greater than 25 mg, to be delivered in a single inhalation and in an advantageous manner. To achieve this, the bulk density of the powder is preferably less than 0.5 g/cm³, and more preferably less than 0.2 g/cm³. Generally, a drug loading of more than 5%, more preferably more than 10%, more preferably more than 20%, more preferably more than 30%, and most preferably more than 40% is also desirable when the required lung dose in more than 5 mg. Alternatively, a dosage may be delivered over two or more inhalations. For example, a 5 mg dosage may be delivered by providing two unit doses of 2.5 mg each, and the two unit doses may be separately aerosolized and inhaled.

The pharmaceutical formulation of the present invention has a substantially improved emitted dose efficiency. Accordingly, high doses of the pharmaceutical formulation may be delivered using a variety of aerosolization devices and techniques. As used herein, the term “emitted dose” or “ED” refers to an indication of the delivery of dry powder from a suitable inhaler device after a firing or dispersion event from a powder unit or reservoir. ED is defined as the ratio of the dose delivered by an inhaler device (described in detail below) to the nominal dose (i.e., the mass of powder per unit dose placed into a suitable inhaler device prior to firing). The ED is an experimentally-determined amount, and is typically determined using an in-vitro device set up which mimics patient dosing. To determine an ED value, a nominal dose of dry powder (as defined above) is placed into a suitable dry powder inhaler, which is then actuated, dispersing the powder. The resulting aerosol cloud is then drawn by vacuum from the device, where it is captured on a tared filter attached to the device mouthpiece. The amount of powder that reaches the filter constitutes the delivered dose. For example, for a 5 mg, dry powder-containing blister pack placed into an inhalation device, if dispersion of the powder results in the recovery of 4 mg of powder on a tared filter as described above, then the ED for the dry powder composition is: 4 mg (delivered dose)/5 mg (nominal dose)×100=80%.

These unit dose pharmaceutical formulations may be contained in a capsule that may be inserted into an aerosolization device. The capsule may be of a suitable shape, size, and material to contain the pharmaceutical formulation and to provide the pharmaceutical formulation in a usable condition. For example, the capsule may comprise a wall which comprises a material that does not adversely react with the pharmaceutical formulation. In addition, the wall may comprise a material that allows the capsule to be opened to allow the pharmaceutical formulation to be aerosolized. In one version, the wall comprises one or more of gelatin, hydroxypropyl methylcellulose (HPMC), polyethyleneglycol-compounded HPMC, hydroxyproplycellulose, agar, or the like. In one version, the capsule may comprise telescopically adjoining sections, as described for example in U.S. Pat. No. 4,247,066 which is incorporated herein by reference in its entirety. The size of the capsule may be selected to adequately contain the dose of the pharmaceutical formulation. The sizes generally range from size 5 to size 000 with the outer diameters ranging from about 4.91 mm to 9.97 mm, the heights ranging from about 11.10 mm to about 26.14 mm, and the volumes ranging from about 0.13 ml to about 1.37 ml, respectively. Suitable capsules are available commercially from, for example, Shionogi Qualicaps Co. in Nara, Japan and Capsugel in Greenwood, S.C. After filling, a top portion may be placed over the bottom portion to form the a capsule shape and to contain the powder within the capsule, as described in U.S. Pat. No. 4,846,876, U.S. Pat. No. 6,357,490, and in the PCT application WO 00/07572 published on Feb. 17, 2000, all of which are incorporated herein by reference in their entireties.

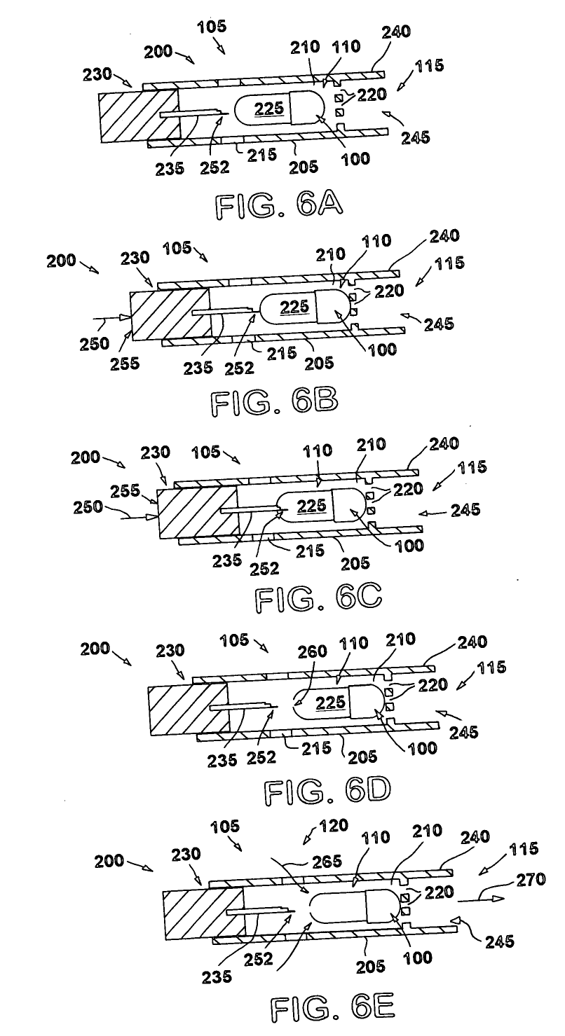

An example of a dry powder aerosolization apparatus particularly useful in aerosolizing a pharmaceutical formulation 100 according to the present invention is shown schematically in FIG. 6A. The aerosolization apparatus 200 comprises a housing 205 defining a chamber 210 having one or more air inlets 215 and one or more air outlets 220. The chamber 210 is sized to receive a capsule 225 which contains an aerosolizable pharmaceutical formulation. A puncturing mechanism 230 comprises a puncture member 235 that is moveable within the chamber 210. Near or adjacent the outlet 220 is an end section 240 that may be sized and shaped to be received in a user's mouth or nose so that the user may inhale through an opening 245 in the end section 240 that is in communication with the outlet 220.

The dry powder aerosolization apparatus 200 utilizes air flowing through the chamber 210 to aerosolize the pharmaceutical formulation in the capsule 225. For example, FIGS. 6A through 6E illustrate the operation of a version of an aerosolization apparatus 200 where air flowing through the inlet 215 is used to aerosolize the pharmaceutical formulation and the aerosolized pharmaceutical formulation flows through the outlet 220 so that it may be delivered to the user through the opening 245 in the end section 240. The dry powder aerosolization apparatus 200 is shown in its initial condition in FIG. 6A. The capsule 225 is positioned within the chamber 210 and the pharmaceutical formulation is contained within the capsule 225.

To use the aerosolization apparatus 200, the pharmaceutical formulation in the capsule 225 is exposed to allow it to be aerosolized. In the version of FIGS. 6A though 6E, the puncture mechanism 230 is advanced within the chamber 210 by applying a force 250 to the puncture mechanism 230. For example, a user may press against a surface 255 of the puncturing mechanism 230 to cause the puncturing mechanism 230 to slide within the housing 205 so that the puncture member 235 contacts the capsule 225 in the chamber 210, as shown in FIG. 6B. By continuing to apply the force 250, the puncture member 235 is advanced into and through the wall of the capsule 225, as shown in FIG. 6C. The puncture member may comprise one or more sharpened tips 252 to facilitate the advancement through the wall of the capsule 225. The puncturing mechanism 230 is then retracted to the position shown in FIG. 6D, leaving an opening 260 through the wall of the capsule 225 to expose the pharmaceutical formulation in the capsule 225.

Air or other gas then flows through an inlet 215, as shown by arrows 265 in FIG. 6E. The flow of air causes the pharmaceutical formulation to be aerosolized. When the user inhales 270 through the end section 240 the aerosolized pharmaceutical formulation is delivered to the user's respiratory tract. In one version, the air flow 265 may be caused by the user's inhalation 270. In another version, compressed air or other gas may be ejected into the inlet 215 to cause the aerosolizing air flow 265.

A specific version of a dry powder aerosolization apparatus 200 is described in U.S. Pat. No. 4,069,819 and in U.S. Pat. No. 4,995,385, both of which are incorporated herein by reference in their entireties. In such an arrangement, the chamber 210 comprises a longitudinal axis that lies generally in the inhalation direction, and the capsule 225 is insertable lengthwise into the chamber 210 so that the capsule's longitudinal axis may be parallel to the longitudinal axis of the chamber 210. The chamber 210 is sized to receive a capsule 225 containing a pharmaceutical formulation in a manner which allows the capsule to move within the chamber 210. The inlets 215 comprise a plurality of tangentially oriented slots. When a user inhales through the endpiece, outside air is caused to flow through the tangential slots. This airflow creates a swirling airflow within the chamber 210. The swirling airflow causes the capsule 225 to contact a partition and then to move within the chamber 210 in a manner that causes the pharmaceutical formulation to exit the capsule 225 and become entrained within the swirling airflow. This version is particularly effective in consistently aerosolizing high doses if the pharmaceutical formulation. In one version, the capsule 225 rotates within the chamber 210 in a manner where the longitudinal axis of the capsule is remains at an angle less than 80 degrees, and preferably less than 45 degrees from the longitudinal axis of the chamber. The movement of the capsule 225 in the chamber 210 may be caused by the width of the chamber 210 being less than the length of the capsule 225. In one specific version, the chamber 210 comprises a tapered section that terminates at an edge. During the flow of swirling air in the chamber 210, the forward end of the capsule 225 contacts and rests on the partition and a sidewall of the capsule 225 contacts the edge and slides and/or rotates along the edge. This motion of the capsule is particularly effective in forcing a large amount of the pharmaceutical formulation through one or more openings 260 in the rear of the capsule 225.

In another passive dry powder inhaler version, the dry powder aerosolization apparatus 200 may be configured differently than as shown in FIGS. 6A through 6E. For example, the chamber 210 may be sized and shaped to receive the capsule 225 so that the capsule 225 is orthogonal to the inhalation direction, as described in U.S. Pat. No. 3,991,761. As also described in U.S. Pat. No. 3,991,761, the puncturing mechanism 230 may puncture both ends of the capsule 225. In another version, the chamber may receive the capsule 225 in a manner where air flows through the capsule 225 as described for example in U.S. Pat. No. 4,338,931 and in U.S. Pat. No. 5,619,985. As used herein, “passive dry powder inhaler” refers to an inhalation device which relies upon the patient's inspiratory effort to disperse and aerosolize a drug formulation contained within the device and does not include inhaler devices which comprise a means for providing energy to disperse and aerosolize the drug formulation, such as pressurized gas and vibrating or rotating elements. In another version, the aerosolization of the pharmaceutical formulation may be accomplished by pressurized gas flowing through the inlets, as described for example in U.S. Pat. No. 5,458,135, U.S. Pat. No. 5,785,049, and U.S. Pat. No. 6,257,233, or propellant, as described in PCT Publication WO 00/72904 and U.S. Pat. No. 4,114,615. These types of dry powder inhalers are generally referred to as active dry powder inhalers. As used herein, “active dry powder inhaler” refers to an inhalation device which does not rely solely on the patient's inspiratory effort to disperse and aerosolize a drug formulation contained within the device and does include inhaler devices which comprise a means for providing energy to disperse and aerosolize the drug formulation, such as pressurized gas and vibrating or rotating elements. All of the above references being incorporated herein by reference in their entireties.

The pharmaceutical formulation disclosed herein may also be administered to the pulmonary air passages of a patient via aerosolization, such as with a metered dose inhaler. The use of such stabilized preparations provides for superior dose reproducibility and improved lung deposition as disclosed in WO 99/16422, which is incorporated herein by reference in its entirety. MDIs are well known in the art and could be employed for administration of the antifungal agent. Breath activated MDIs, as well as those comprising other types of improvements which have been, or will be, developed are also compatible with the pharmaceutical formulation of the present invention.

Along with the aforementioned embodiments, the stabilized dispersions of the present invention may also be used in conjunction with nebulizers as disclosed in PCT WO 99/16420, the disclosure of which is incorporated herein by reference in its entirety, in order to provide an aerosolized medicament that may be administered to the pulmonary air passages of a patient in need thereof. Nebulizers are well known in the art and could easily be employed for administration of the claimed dispersions without undue experimentation. Breath activated nebulizers, as well as those comprising other types of improvements which have been, or will be, developed are also compatible with the stabilized dispersions and present invention and are contemplated as being with in the scope thereof.

Along with DPIs, MDIs and nebulizers, it will be appreciated that the stabilized dispersions of the present invention may be used in conjunction with liquid dose instillation or LDI techniques as disclosed in, for example, WO 99/16421 which is incorporated herein by reference in its entirety. Liquid dose instillation involves the direct administration of a stabilized dispersion to the lung. In this regard, direct pulmonary administration of bioactive compounds is particularly effective in the treatment of disorders especially where poor vascular circulation of diseased portions of a lung reduces the effectiveness of intravenous drug delivery. With respect to LDI the stabilized dispersions are preferably used in conjunction with partial liquid ventilation or total liquid ventilation. Moreover, the present invention may further comprise introducing a therapeutically beneficial amount of a physiologically acceptable gas (such as nitric oxide or oxygen) into the pharmaceutical microdispersion prior to, during or following administration.

It will be appreciated that the particulate compositions disclosed herein comprise a structural matrix that exhibits, defines or comprises voids, pores, defects, hollows, spaces, interstitial spaces, apertures, perforations or holes. The absolute shape (as opposed to the morphology) of the perforated microstructure is generally not critical and any overall configuration that provides the desired characteristics is contemplated as being within the scope of the invention. Accordingly, preferred embodiments can comprise approximately microspherical shapes. However, collapsed, deformed or fractured particulates are also compatible.

In accordance with the teachings herein the particulate compositions will preferably be provided in a “dry” state. That is the particulates will possess a moisture content that allows the powder to remain chemically and physically stable during storage at ambient temperature and easily dispersible. As such, the moisture content of the microparticles is typically less than 6% by weight, and preferably less 3% by weight. In some instances the moisture content will be as low as 1% by weight. The moisture content is, at least in part, dictated by the formulation and is controlled by the process conditions employed, e.g., inlet temperature, feed concentration, pump rate, and blowing agent type, concentration and post drying. Reduction in bound water leads to significant improvements in the dispersibility and flowability of phospholipid based powders, leading to the potential for highly efficient delivery of powdered lung surfactants or particulate composition comprising active agent dispersed in the phospholipid. The improved dispersibility allows simple passive DPI devices to be used to effectively deliver these powders.

Although the powder compositions are preferably used for inhalation therapies, the powders of the present invention can also be administered by other techniques known in the art, including, but not limited to oral, intramuscular, intravenous, intratracheal, intraperitoneal, subcutaneous, and transdermal, either as capsules, tablets, dry powders, reconstituted powders, or suspensions.

According to another embodiment, release kinetics of the active agent containing composition is controlled. According to a preferred embodiment, the compositions of the present invention provide immediate release due to the size or amount of the antifungal agent incorporated into the matrix material. Alternatively, the compositions of the present invention may be provided as non-homogeneous mixtures of active agent incorporated into a matrix material and unincorporated active agent in order to provide desirable release rates of antifungal agent. According to this embodiment, antifungal agents formulated using the emulsion-based manufacturing process of the present invention have utility in immediate release applications when administered to the respiratory tract. Rapid release is facilitated by: (a) the high surface area of the low density porous powders; (b) the small size of the drug crystals that are incorporated therein, and; (c) the low surface energy of the particles resulting from the lack of long-range order for the phospholipids on the surface of the particles.

Alternatively, it may be desirable to engineer the particle matrix so that extended release of the antifungal agent is effected. This may be particularly desirable when the antifungal agent is rapidly cleared from the lungs. For example, the nature of the surface packing of phospholipid molecules is influenced by the nature of their packing in the spray-drying feedstock and the drying conditions and other formulation components utilized. In the case of spray-drying of active agents solubilized within a small unilamellar vesicle (SUV) or multilamellar vesicle (MLV), the active remains encapsulated within multiple bilayers with a high degree of long-range order over fairly large length scales. In this case, the spray-dried formulation may exhibit sustained release characteristics.

In contrast, spray-drying of a feedstock comprised of emulsion droplets and dispersed or dissolved active in accordance with the teachings herein leads to a phospholipid matrix with less long-range order, thereby facilitating rapid release. While not being bound to any particular theory, it is believed that this is due in part to the fact that the active is never formally encapsulated in the phospholipid, and the fact that the phospholipid is initially present on the surface of the emulsion droplets as a monolayer (not a bilayer as in the case of liposomes). The higher degree of disorder observed in spray-dried particles prepared by the emulsion-based manufacturing process of the present invention is reflected in very low surface energies, where values as low as 20 mN/m have been observed for spray-dried DSPC particles (determined by inverse gas chromatography). Small angle X-ray scattering (SAXS) studies conducted with spray-dried phospholipid particles have also shown a high degree of disorder for the lipid, with scattering peaks smeared out, and length scales extending in some instances only beyond a few nearest neighbors.

It should be noted that having a high gel to liquid crystal phase transition temperature is not sufficient in itself in achieving sustained release. Having a sufficient length scale for the bilayer structures is also important. To facilitate rapid release, an emulsion-system of high porosity (high surface area), and no interaction between the drug substance and phospholipid is preferred. The pharmaceutical formulation formation process may also include the additions of other formulation components (e.g., small polymers such as Pluronic F-68; carbohydrates, salts, hydrotropes) to break the bilayer structure are also contemplated.

To achieve a sustained release, incorporation of the phospholipid in bilayer form is preferred, especially if the active agent is encapsulated therein. In this case increasing the Tm of the phospholipid may provide benefit via incorporation of divalent counterions or cholesterol. As well, increasing the interaction between the phospholipid and drug substance via the formation of ion-pairs (negatively charged active+steaylamine, postitively charged active+phosphatidylglycerol) would tend to decrease the dissolution rate. If the active is amphiphilic, surfactant/surfactant interactions may also slow active dissolution.

The addition of divalent counterions (e.g. calcium or magnesium ions) to long-chain saturated phosphatidylcholines results in an interaction between the negatively charged phosphate portion of the zwitterionic headgroup and the positively charged metal ion. This results in a displacement of water of hydration and a condensation of the packing of the phospholipid lipid headgroup and acyl chains. Further, this results in an increase in the Tm of the phospholipid. The decreases in headgroup hydration can have profound effects on the spreading properties of spray-dried phospholipid particles on contact with water. A fully hydrated phosphatidylcholine molecule will diffuse very slowly to a dispersed crystal via molecular diffusion through the water phase. The process is exceedingly slow because the solubility of the phospholipid in water is very low (ca.,10⁻¹⁰ mol/L for DPPC). Prior art attempts to overcome this phenomena include homogenizing the crystals in the presence of the phospholipid. In this case, the high degree of shear and radius of curvature of the homogenized crystals facilitates coating of the phospholipid on the crystals. In contrast, “dry” phospholipid powders according to this invention can spread rapidly when contacted with an aqueous phase, thereby coating dispersed crystals without the need to apply high energies. For example, the surface tension of spray-dried DSPC/Ca mixtures at the air/water interface decreases to equilibrium values (ca., 20 mN/m) as fast as a measurement can be taken. In contrast, liposomes of DSPC decrease the surface tension (ca., 50 mN/m) very little over a period of hours, and it is likely that this reduction is due to the presence of hydrolysis degradation products such as free fatty acids in the phospholipid. Single-tailed fatty acids can diffuse much more rapidly to the air/water interface than can the hydrophobic parent compound. Hence the addition of calcium ions to phosphatidylcholines can facilitate the rapid encapsulation of crystalline drugs more rapidly and with the lower applied energy.

In another version, the pharmaceutical formulation comprises low density particulates achieved by co-spray-drying nanocrystals with a perfluorocarbon-in-water emulsion.

The foregoing description will be more fully understood with reference to the following Examples. Such Examples, are, however, merely representative of preferred methods of practicing the present invention and should not be read as limiting the scope of the invention.

### EXAMPLE I

Preparation of Spray-Dried Amphotericin B Particles

Amphotericin particles were prepared by a two-step process. In the first step, 10.52 g of amphotericin B (Alpharma, Copenhagen, Denmark), 10.12 g of distearoyl phosphatidylcholine (DSPC) (Genzyme, Cambridge, Mass.), and 0.84 g calcium chloride (JT Baker, Phillipsburg, N.J.) were dispersed in 1045 g of hot deionized water (T=70° C.) using an Ultra-Turrax mixer (model T-25) at 10,000 rpm for 2 to 5 minutes. Mixing was continued until the DSPC and amphotericin B appeared visually to be dispersed.

381 g of perfluorooctyl ethane (PFOE) was then added slowly at a rate of approximately 50-60 ml/min during mixing After the addition was complete, the emulsion/drug dispersion was mixed for an additional period of not less than 5 minutes at 12,000 rpm. The coarse emulsion was then passed through a high pressure homogenizer (Avestin, Ottawa, Canada) at 12,000-18,000 psi for 3 passes, followed by 2 passes at 20,000-23,000 psi.

The resulting fine emulsion was utilized as the feedstock in for the second step, i.e. spray-drying on a Niro Mobile Minor. The following spray conditions were employed: total flow rate=70 SCFM, inlet temperature=110° C., outlet temperature=57° C., feed pump=38 mL min⁻¹, atomizer pressure=105 psig, atomizer flow rate=12 SCFM.

A free flowing pale yellow powder was collected using a cyclone separator. The collection efficiency of the amphotericin B formulation was 60%. The geometric diameter of the amphotericin B particles was confirmed by laser diffraction (Sympatech Helos H1006, Clausthal-Zellerfeld, Germany), where a volume weighted mean diameter (VMD) of 2.44 μm was found. Scanning electron microscopy (SEM) analysis showed the powders to be small porous particles with high surface roughness. There was no evidence of any unincorporated AmB crystals in the 5 SEM views provided for each collector. Differential scanning calorimetry analysis of the dry particles revealed the tₘ for the amphotericin B in the powder to be 78° C., which is similar to what is observed for spray-dried neat material.

### EXAMPLE II

Aerosol Performance for Spray-Dried Amphotericin B Particles

The resulting dry amphotericin B particles prepared in Example I were hand filled into #2 HPMC capsules (Shionogi, Japan) and allowed to equilibrate at 15%-20% RH overnight. A fill mass of approximately 10 mg was used, which represented approximately ½ the fill volume of the #2 capsule.

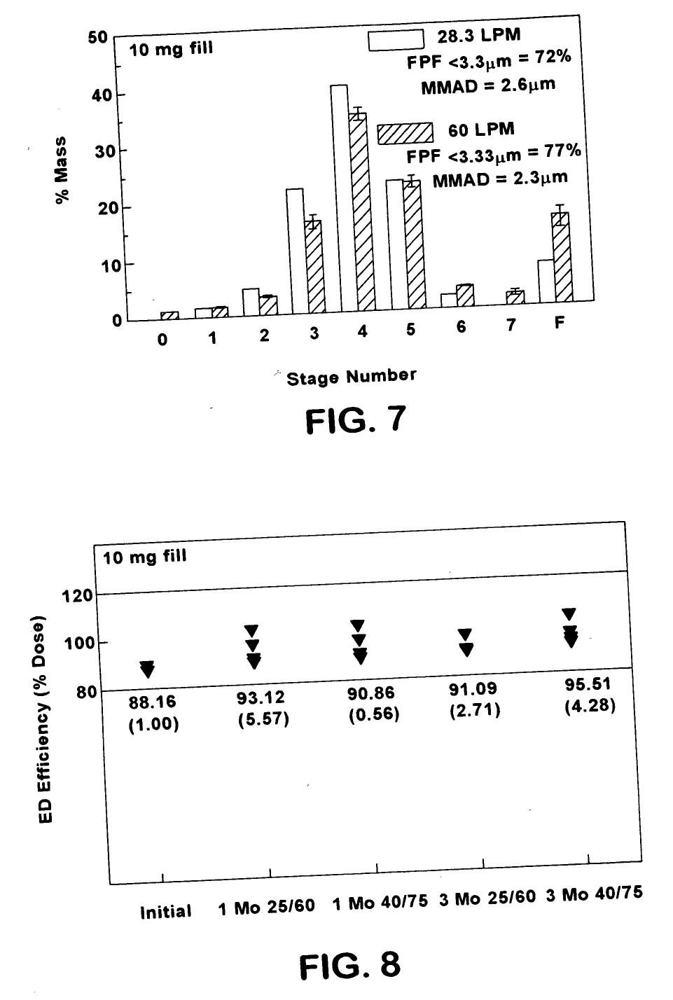

Aerodynamic particle size distributions were determined gravimetrically on an Andersen cascade impactor (ACI). Particle size distributions were measured at flow rates of 28.3 L·min⁻¹ (i.e., comfortable inhalation effort) and 56.6 L·min⁻¹ (i.e., forceful inhalation effort) using the Turbospin DPI device described in U.S. Pat. Nos. 4,069,819 and 4,995,385, both of which are incorporated herein by reference in their entireties. A total volume of 2 liters was drawn through the device. At the higher flow rate, two ACIs were used in parallel at a calibrated flow rate of 28.3 L·min⁻¹ and a total flow through the device of 56.6 L·min⁻¹. In both cases the set-up represents conditions at which the ACI impactor plates are calibrated. Excellent aerosol characteristics was observed as evidenced by a MMAD less than 2.6 μm and FPF_{<3.3 μm} greater than 72%. The effect of flow rate on performance was also assessed (FIG. 7) using the Turbospin® (PH&T, Italy) DPI device operated at 56.6 L min⁻¹ into 2 ACIs used in parallel. No significant difference in the deposition profile was observed at the higher flow rates, demonstrating minimal flow rate dependant performance. This abovementioned example illustrates the aerosol performance of the present powder is independent of flow rate which should lead to more reproducible patient dosing.

### EXAMPLE III

Effect of Stability Storage on Aerosol Performance of Spray-Dried Amphotericin B Particles

The resulting dry amphotericin B particles prepared in Example I were hand filled into #2 HPMC capsules (Shionogi, Japan) and allowed to equilibrate at 15%-20% RH overnight. A fill mass of approximately 10 mg was used, which represented approximately ½ the fill volume of the #2 capsule. The filled capsules were placed in individually indexed glass vials that were packaged in laminated foil-sealed pouch and subsequently stored at 25° C./60% RH or 40° C./75% RH.

Emitted dose (ED) measurements were performed using the Turbospin® (PH&T, Italy) DPI device, described in U.S. Pat. No. 4,069,819 and in U.S. Pat. No. 4,995,385, operated at its optimal sampling flow rate of 60 L·min⁻¹, and using a total volume of 2 liters. A total of 10 measurements was determined for each storage variant.

The aerodynamic particle size distributions were determined gravimetrically on an Andersen cascade impactor (ACI). Particle size distributions were measured at flow rates of 28.3 L·min⁻¹ using the Turbospin® DPI device and using a total volume of 2 liters.

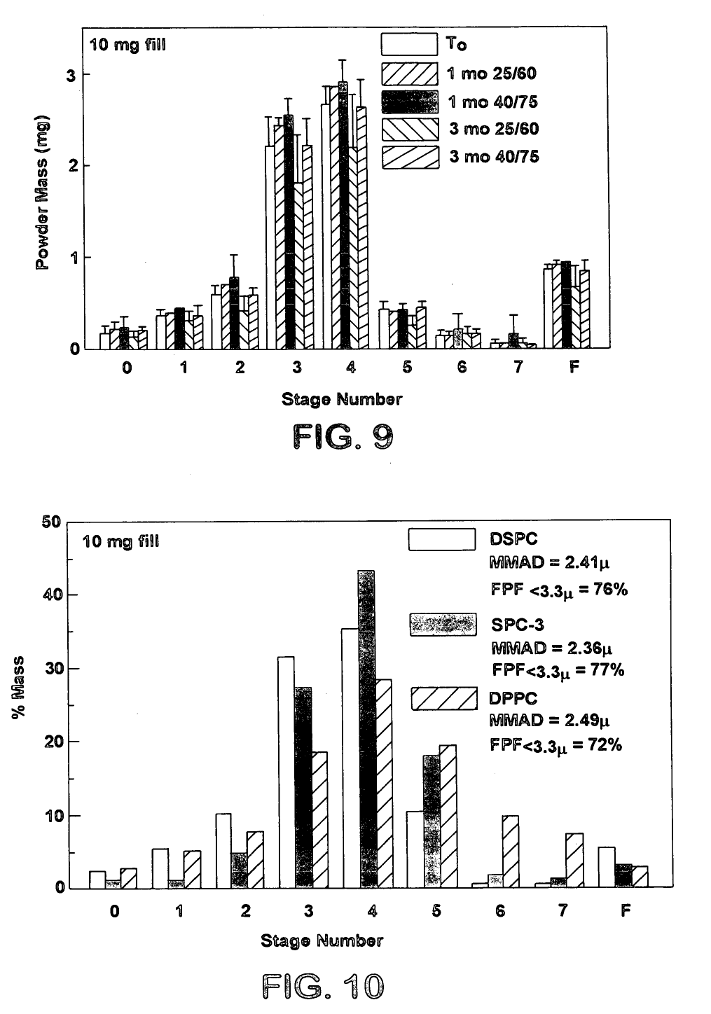

Excellent aerosol characteristics was observed as evidenced by a mean ED of 93%+/−5.3%, MMAD=2.6 μm and FPF_{<3.3 μm}=72% (FIGS. 8 and 9). No significant change in aerosol performance (ED, MMAD or FPF) was observed after storage at elevated temperature and humidity, demonstrating excellent stability characteristics. The current specifications ED performance stipulates that >90% of the delivered doses be within ±25% of the label claim, with less than 10% of the doses ±35%. A recent draft guidance published by the FDA [10] proposes that the limits be tightened, such that >90% of the delivered doses be within ±20% of the label claim, with none outside of ±25%. Statistically speaking, an RSD of 6% would be required to meet the proposed FDA specifications.

Not only are the results of the foregoing example within the current guidelines, but they are also within the limits of the proposed guidelines, a strong testament to the excellent dispersibility, aerosol characteristics and stability afforded by this formulation.

### EXAMPLE IV

Spray-Dried Amphotericin B Particles Comprised of Various Phosphatidylcholines

Spray-dried particles comprising approximately 50% amphotericin B were prepared using various phosphatidylcholines (PC) as the surfactant following the two-step process described in Example I. Formulations were prepared using DPPC (Genzyme, Cambridge, Mass.), DSPC (Genzyme, Cambridge, Mass.) and SPC-3 (Lipoid KG, Ludwigshafen, Germany) The feed solution was prepared using the identical equipment and process conditions described therein. The 50% amphotericn B formulation is as follows:

| | |
|---|---|
| Amphotericin B | 0.733 g |
| PC | 0.714 g |
| CaCl₂ | 60 mg |
| PFOB | 32 g |
| DI water | 75 g |

The resulting multi-particulate emulsion was utilized as the feedstock in for the second step, i.e. spray-drying on a B-191 Mini Spray-Drier (Büchi, Flawil, Switzerland). The following spray conditions were employed: aspiration=100%, inlet temperature=85° C., outlet temperature=60° C., feed pump=1.9 mL min⁻¹, atomizer pressure=60-65 psig, atomizer flow rate=30-35 cm. The aspiration flow (69-75%) was adjusted to maintain an exhaust bag pressure of 30-31 mbar. Free flowing yellow powders were collected using a standard cyclone separator. The geometric diameter of the amphotericin B particles was confirmed by laser diffraction (Sympatech Helos H1006, Clausthal-Zellerfeld, Germany), where a volume weighted mean diameters (VMD) were found to be similar and ranged from 2.65 μm to 2.75 μm. Scanning electron microscopy (SEM) analysis showed the powders to be small porous particles with high surface roughness.

Aerodynamic particle size distributions were determined gravimetrically on an Andersen cascade impactor (ACI), see FIG. 10. Particle size distributions were measured at flow rates of 56.6 L·min⁻¹ (i.e., forceful inhalation effort) using the Turbospin DPI device. A total volume of 2 liters was drawn through the device. Two ACIs were used in parallel at a calibrated flow rate of 28.3 L·min⁻¹ and a total flow through the devices of 56.6 L·min⁻¹. Similar aerosol characteristics were observed in the amphotericin B produced with the 3 types of phosphatidylcholines, with MMADs less than 2.5 μm and FPF_{<3.3 μm} greater than 72%. This abovementioned example illustrates the flexibility of the formulation technology to produce amphotericin B powders independent of the type of phosphatidylcholie employed.

### EXAMPLE V

Preparation of 70% Amphotericin B Spray-Dried Particles.

Amphotericin particles were prepared following the two-step process described in Example I. The feed solution was prepared using the identical equipment and process conditions described therein. The 70% amphotericn B formulation is as follows:

| | |
|---|---|
| Amphotericin B | 0.70 g |
| DSPC | 0.265 g |
| CaCl₂ | 24 mg |
| PFOB | 12 g |
| DI water | 35 g |

The resulting multi-particulate emulsion was utilized as the feedstock in for the second step, i.e. spray-drying on a B-191 Mini Spray-Drier (Büchi, Flawil, Switzerland). The following spray conditions were employed: aspiration=100%, inlet temperature=85° C., outlet temperature=60° C., feed pump=1.9 mL min⁻¹, atomizer pressure=60-65 psig, atomizer flow rate=30-35 cm. The aspiration flow (69-75%) was adjusted to maintain an exhaust bag pressure of 30-31 mbar. A free flowing yellow powder was collected using a standard cyclone separator. The geometric diameter of the amphotericin B particles was confirmed by laser diffraction (Sympatech Helos H1006, Clausthal-Zellerfeld, Germany), where a volume weighted mean diameter (VMD) of 2.96 μm was found. Scanning electron microscopy (SEM) analysis showed the powders to be small porous particles with high surface roughness. This foregoing example illustrates the flexibility of the present powder engineering technology to produce high amphotericin B content using the herein described multi-particulate approach.

### EXAMPLE VI

Aerosol Performance of Spray-Dried Amphotericin B Particles in Various DPI Devices.

The resulting dry amphotericin B particles prepared in Example V were hand filled into #2 HPMC (Shionogi, Japan) or #3 (Capsugel, Greenwood, S.C.) capsules and allowed to equilibrate at 15%-20% RH overnight. A fill mass of approximately 10 mg was used, which represents approximately ½ the fill volume for a #2 capsule or 5/8 for a #3 capsule. The aerosol characteristics were examined using a Turbospin® (PH&T, Italy), Eclipse® (Aventis, UK) and Cyclohaler® (Novartis, Switzerland) DPI devices. The Cyclohaler utilizes a # 3 capsule, whereas the Turbospin and Cyclohaler utilize size # 2 capsules

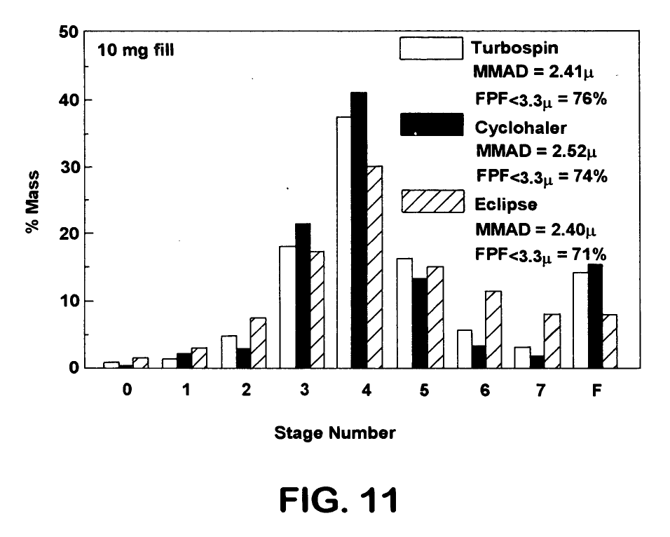

Aerodynamic particle size distributions were determined gravimetrically on an Andersen cascade impactor (ACI), see FIG. 11. Particle size distributions were measured at a flow rate 56.6 L·min⁻¹ which represents a forceful inhalation effort for both Turbospin and Eclipse DPI devices and comfortable for Cyclohaler. A total volume of 2 liters was drawn through the device. Two ACIs were used in parallel at a calibrated flow rate of 28.3 L·min⁻¹ and a total flow through the devices of 56.6 L·min⁻¹. Similar aerosol characteristics were observed in all devices as evidenced by a MMAD less than 2.5 μm and FPF_{<3.3 μm} greater than 71%. This above-mentioned example illustrates the aerosol performance of the present powder is independent of device design with medium and low resistance and capsule size speaks volumes to the dispersibility of the amphotericin B powder tested.

Although the present invention has been described in considerable detail with regard to certain preferred versions thereof, other versions are possible, and alterations, permutations and equivalents of the version shown will become apparent to those skilled in the art upon a reading of the specification and study of the drawings. For example, the relative positions of the elements in the aerosolization device may be changed, and flexible parts may be replaced by more rigid parts that are hinged, or otherwise movable, to mimic the action of the flexible part. In addition, the passageways need not necessarily be substantially linear, as shown in the drawings, but may be curved or angled, for example. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

What is claimed is:

1. A method of treating and/or providing prophylaxis against a pulmonary fungal infection, the method comprising:
determining the minimum inhibitory concentration of an antifungal agent for inhibiting pulmonary fungal growth; and
administering an aerosolized pharmaceutical formulation comprising the antifungal agent to the lungs of a patient;
wherein a sufficient amount of the pharmaceutical formulation is administered to maintain for at least one week a target antifungal agent lung concentration of at least two times the determined minimum inhibitory concentration.

2. A method according to claim 1 wherein the minimum inhibitory concentration is the minimum inhibitory concentration in the epithelial lining of the lung.

3. A method according to claim 1 wherein the minimum inhibitory concentration is the minimum inhibitory concentration in the solid tissue of the lung.

4. A method according to claim 1 wherein the target antifungal agent lung concentration is maintained for at least two weeks.

5. A method according to claim 1 wherein the target antifungal agent lung concentration is maintained for at least three weeks.

6. A method according to claim 1 wherein the target antifungal agent lung concentration is maintained for at least one month.

7. A method according to claim 1 wherein the target antifungal agent lung concentration is maintained for at least three months.

8. A method according to claim 1 wherein the administration comprises delivering a single dose of the pharmaceutical formulation during the first week of administration.

9. A method according to claim 1 wherein the administration comprises delivering at least two doses of the pharmaceutical formulation during the first week of administration.

10. A method according to claim 1 wherein the administration comprises a first administration period and a second administration period and wherein the antifungal agent is administered more frequently or at a higher dosage during the first administration period than during the second administration period.

11. A method according to claim 1 wherein the antifungal agent is amphotericin B.

12. A method according to claim 11 wherein the target antifungal lung concentration is at least 9 μg/g.

13. A method according to claim 11 wherein the target antifungal lung concentration is a range of concentrations from 4.5 μg/g to 20 μg/g and wherein the administration comprises delivering the pharmaceutical formulation periodically to maintain the antifungal agent lung concentration within the target antifungal lung concentration range.

14. A method according to claim 13 wherein the target antifungal lung concentration is from 9 to 15 μg/g.

15. A method according to claim 1 wherein the antifungal agent comprises one or more of amphotericin B, nystatin, hamycin, natamycin, pimaricin, ambruticin, acrisocin, aminacrine, anthralin, benanomicin A, benzoic acid, butylparaben, calcium unidecyleneate, candicidin, ciclopirox olamine, cilofungin, clioquinol, clotrimazole, ecaonazole, flucanazole, flucytosine, gentian violet, griseofulvin, haloprogin, ichthammol, iodine, itraconazole, ketoconazole, voriconazole, miconazole, nikkomycin Z, potassium iodide, potassium permanganate, pradimicin A, propylparaben, resorcinol, sodium benzoate, sodium propionate, sulconazole, terconazole, tolnaftate, triacetin, unidecyleneic acid, monocyte-macrophage colony stimulating factor (M-CSF), zinc unidecylenateand, and pharmaceutically acceptable derivatives and salts thereof.

16. A method according to claim 1 wherein the pharmaceutical formulation has a bulk density of less than 0.5 g/cm³.

17. A method according to claim 1 wherein the pharmaceutical formulation comprises hollow and/or porous particles.

18. A method according to claim 1 wherein the pharmaceutical formulation comprises particles comprising the antifungal agent and a matrix material.

19. A method according to claim 18 wherein the matrix material comprises one or more phospholipids.

20. A method according to claim 1 wherein the administration comprises delivering the pharmaceutical formulation in dry powder form using a dry powder inhaler.

21. A method according to claim 1 wherein the pharmaceutical formulation comprises a propellant and wherein the administration comprises aerosolizing the antifungal agent by opening a valve to release the pharmaceutical formulation.

22. A method according to claim 1 wherein the pharmaceutical formulation is a liquid and wherein the administration comprises aerosolizing the liquid using a compressed gas and/or a vibrating member.

23. A method of treating and/or providing prophylaxis against a pulmonary fungal infection, the method comprising:
administering an aerosolized pharmaceutical formulation comprising amphotericin B to the lungs of a patient;
wherein a sufficient amount of the pharmaceutical formulation is administered to maintain for at least one week a target amphotericin lung concentration of at least 5 μg/g.

24. A method according to claim 23 wherein the amphotericin B concentration is the concentration in the epithelial lining of the lung.

25. A method according to claim 23 wherein the amphotericin B concentration is the concentration in the solid tissue of the lung.

26. A method according to claim 23 wherein the target amphotericin B lung concentration is maintained for at least two weeks.

27. A method according to claim 23 wherein the target amphotericin B lung concentration is maintained for at least three weeks.

28. A method according to claim 23 wherein the target amphotericin B lung concentration is maintained for at least one month.

29. A method according to claim 23 wherein the target amphotericin B lung concentration is maintained for at least three months.

30. A method according to claim 23 wherein the administration comprises delivering a single dose of the pharmaceutical formulation during the first week of administration.

31. A method according to claim 23 wherein the administration comprises delivering at least two doses of the pharmaceutical formulation during the first week of administration.

32. A method according to claim 23 wherein the administration comprises a first administration period and a second administration period and wherein the amphotericin B is administered more frequently or at a higher dosage during the first administration period than during the second administration period.

33. A method according to claim 23 wherein the target amphotericin B lung concentration is at least 9 μg/g.

34. A method according to claim 23 wherein the target amphotericin B lung concentration is a range of concentrations from 5 μg/g to 20 μg/g and wherein the administration comprises delivering the pharmaceutical formulation periodically to maintain the amphotericin B lung concentration within the target amphotericin B lung concentration range.

35. A method according to claim 23 wherein the target amphotericin B lung concentration is from 9 to 15 μg/g.

36. A method according to claim 23 wherein the pharmaceutical formulation has a bulk density of less than 0.5 g/cm³.

37. A method according to claim 23 wherein the pharmaceutical formulation comprises hollow and/or porous particles.

38. A method according to claim 23 wherein the pharmaceutical formulation comprises particles comprising the antifungal agent and a matrix material.

39. A method according to claim 38 wherein the matrix material comprises one or more phospholipids.

40. A method according to claim 23 wherein the administration comprises delivering the pharmaceutical formulation in dry powder form using a dry powder inhaler.

41. A method according to claim 23 wherein the pharmaceutical formulation comprises a propellant and wherein the administration comprises aerosolizing the amphotericin B by opening a valve to release the pharmaceutical formulation.

42. A method according to claim 23 wherein the pharmaceutical formulation is a liquid and wherein the administration comprises aerosolizing the liquid using a compressed gas and/or a vibrating member.

43. A method of treating or providing prophylaxis against a pulmonary lung infection, the method comprising:
determining the minimum inhibitory concentration of an antifungal agent for inhibiting pulmonary fungal growth; and
administering at least once per week an aerosolized pharmaceutical formulation comprising the antifungal agent to the lungs of a patient;
wherein the amount of the pharmaceutical formulation administered is sufficient to maintain for at least three weeks a target antifungal agent lung concentration that is greater than the determined minimum inhibitory concentration.

44. A method according to claim 43 wherein the pharmaceutical formulation is administered more than once per week for a first period and is delivered once per week for a second period.

45. A method according to claim 43 wherein the pharmaceutical formulation is administered once per week.

46. A method according to claim 43 wherein the minimum inhibitory concentration is the minimum inhibitory concentration in the epithelial lining of the lung.

47. A method according to claim 43 wherein the minimum inhibitory concentration is the minimum inhibitory concentration in the solid tissue of the lung.

48. A method according to claim 43 wherein the target antifungal agent lung concentration is maintained for at least three months.

49. A method according to claim 43 wherein the antifungal agent is amphotericin B.

50. A method according to claim 49 wherein the target antifungal lung concentration is at least 9 μg/g.

51. A method according to claim 49 wherein the target antifungal lung concentration is from 9 to 15 ˜g/g.

52. A method according to claim 43 wherein the antifungal agent comprises one or more of amphotericin B, nystatin, hamycin, natamycin, pimaricin, ambruticin, acrisocin, aminacrine, anthralin, benanomicin A, benzoic acid, butylparaben, calcium unidecyleneate, candicidin, ciclopirox olamine, cilofungin, clioquinol, clotrimazole, ecaonazole, flucanazole, flucytosine, gentian violet, griseofulvin, haloprogin, ichthammol, iodine, itraconazole, ketoconazole, voriconazole, miconazole, nikkomycin Z, potassium iodide, potassium permanganate, pradimicin A, propylparaben, resorcinol, sodium benzoate, sodium propionate, sulconazole, terconazole, tolnaftate, triacetin, unidecyleneic acid, monocyte-macrophage colony stimulating factor (M-CSF), zinc unidecylenateand, and pharmaceutically acceptable derivatives and salts thereof.

53. A method according to claim 43 wherein the pharmaceutical formulation has a bulk density of less than 0.5 g/cm³.

54. A method of treating or providing prophylaxis against a pulmonary lung infection, the method comprising:
administering at least once per week an aerosolized pharmaceutical formulation comprising amphotericin B to the lungs of a patient;
wherein the amount of the pharmaceutical formulation administered is sufficient to maintain for at least three weeks a target amphotericin B lung concentration that is greater than the 4 μg/g.

55. A method according to claim 54 wherein the pharmaceutical formulation is administered more than once per week for a first period and is delivered once per week for a second period.

56. A method according to claim 54 wherein the pharmaceutical formulation is administered once per week.

57. A method according to claim 54 wherein the amphotericin B concentration is the concentration in the epithelial lining of the lung.

58. A method according to claim 54 wherein the amphotericin B concentration is the concentration in the solid tissue of the lung.

59. A method according to claim 54 wherein the target amphotericin B lung concentration is maintained for at least three months.

60. A method according to claim 54 wherein the target amphotericin B lung concentration is at least 9 μg/g.

61. A method according to claim 54 wherein the target amphotericin B lung concentration is from 9 to 15 μg/g.

62. A method according to claim 54 wherein the pharmaceutical formulation has a bulk density of less than 0.5 g/cm³.

63. A method of providing prophylaxis against a pulmonary lung infection, the method comprising:
determining the minimum inhibitory concentration of an antifungal agent for inhibiting pulmonary fungal growth;
administering an aerosolized pharmaceutical formulation comprising the antifungal agent to the lungs of a patient, wherein the amount of the pharmaceutical formulation administered is sufficient to achieve a target antifungal agent lung concentration that is greater than the determined minimum inhibitory concentration;
thereafter administering an immunosuppressive agent to the patient for a period of time; and
maintaining the target antifungal agent lung concentration throughout the period of time.

64. A method according to claim 63 wherein the minimum inhibitory concentration is the minimum inhibitory concentration in the epithelial lining of the lung.

65. A method according to claim 63 wherein the minimum inhibitory concentration is the minimum inhibitory concentration in the solid tissue of the lung.

66. A method according to claim 63 wherein the administration comprises delivering at least two doses per week of the pharmaceutical formulation before the administration of the immunosuppressive agent and wherein the target concentration is maintained by administering doses of the pharmaceutical formulation less frequently.

67. A method according to claim 63 wherein the antifungal agent is amphotericin B.

68. A method according to claim 67 wherein the target antifungal lung concentration is at least 4.5 μg/g.

69. A method according to claim 67 wherein the target antifungal lung concentration is a range of concentrations from 4.5 μg/g to 20 μg/g and wherein the administration comprises delivering the pharmaceutical formulation periodically to maintain the antifungal agent lung concentration within the target antifungal lung concentration range.

70. A method according to claim 67 wherein the target antifungal lung concentration is from 9 to 15 μg/g.

71. A method according to claim 63 wherein the antifungal agent comprises one or more of amphotericin B, nystatin, hamycin, natamycin, pimaricin, ambruticin, acrisocin, aminacrine, anthralin, benanomicin A, benzoic acid, butylparaben, calcium unidecyleneate, candicidin, ciclopirox olamine, cilofungin, clioquinol, clotrimazole, ecaonazole, flucanazole, flucytosine, gentian violet, griseofulvin, haloprogin, ichthammol, iodine, itraconazole, ketoconazole, voriconazole, miconazole, nikkomycin Z, potassium iodide, potassium permanganate, pradimicin A, propylparaben, resorcinol, sodium benzoate, sodium propionate, sulconazole, terconazole, tolnaftate, triacetin, unidecyleneic acid, monocyte-macrophage colony stimulating factor (M-CSF), zinc unidecylenateand, and pharmaceutically acceptable derivatives and salts thereof.

72. A method according to claim 63 wherein the pharmaceutical formulation has a bulk density of less than 0.5 g/cm³.

73. A method according to claim 63 wherein the pharmaceutical formulation comprises hollow and/or porous particles.

74. A method according to claim 63 wherein the pharmaceutical formulation comprises particles comprising the antifungal agent and a matrix material.

75. A method according to claim 74 wherein the matrix material comprises one or more phospholipids.

76. A method according to claim 63 wherein the administration comprises delivering the pharmaceutical formulation in dry powder form using a dry powder inhaler.

77. A method according to claim 63 wherein the pharmaceutical formulation comprises a propellant and wherein the administration comprises aerosolizing the antifungal agent by opening a valve to release the pharmaceutical formulation.

78. A method according to claim 63 wherein the pharmaceutical formulation is a liquid and wherein the administration comprises aerosolizing the liquid using a compressed gas and/or a vibrating member.

79. A method of providing prophylaxis against a pulmonary lung infection, the method comprising:
administering an aerosolized pharmaceutical formulation comprising amphotericin B to the lungs of a patient, wherein the amount of the pharmaceutical formulation administered is sufficient to deliver at least 5 mg of amphotericin B to the lungs per week;
thereafter administering an immunosuppressive agent to the patient for a period of time; and
administering at least 5 mg of amphotericin B to the lungs per week throughout the period of time.

80. A method according to claim 79 wherein the administration comprises delivering at least 10 mg of amphotericin B before the administration of the immunosuppressive agent and delivering a lesser amount per week during the period of immunosuppression.

81. A method according to claim 79 wherein the amount of amphotericin B administered during the period of immunosuppression is from 5 mg to 10 mg.

82. A method according to claim 79 wherein the pharmaceutical formulation has a bulk density of less than 0.5 g/cm³.

83. A method according to claim 79 wherein the pharmaceutical formulation comprises hollow and/or porous particles.

84. A method according to claim 79 wherein the pharmaceutical formulation comprises particles comprising the antifungal agent and a matrix material.

85. A method according to claim 79 wherein the matrix material comprises one or more phospholipids.

86. A method according to claim 79 wherein the administration comprises delivering the pharmaceutical formulation in dry powder form using a dry powder inhaler.

87. A method according to claim 79 wherein the pharmaceutical formulation comprises a propellant and wherein the administration comprises aerosolizing the antifungal agent by opening a valve to release the pharmaceutical formulation.

88. A method according to claim 79 wherein the pharmaceutical formulation is a liquid and wherein the administration comprises aerosolizing the liquid using a compressed gas and/or a vibrating member.

89. A method of treating or providing prophylaxis against a pulmonary lung infection, the method comprising:
delivering an aerosolized pharmaceutical formulation comprising from 5 mg to 10 mg of amphotericin B to the respiratory tract of a patient once per week for a period of at least two weeks.

90. A method according to claim 89 wherein the pharmaceutical formulation has a bulk density of less than 0.5 g/cm³.

91. A method according to claim 89 wherein the pharmaceutical formulation comprises hollow and/or porous particles.

92. A method according to claim 89 wherein the pharmaceutical formulation comprises particles comprising the antifungal agent and a matrix material.

93. A method according to claim 89 wherein the matrix material comprises one or more phospholipids.

94. A method according to claim 89 wherein the administration comprises delivering the pharmaceutical formulation in dry powder form using a dry powder inhaler.

95. A method according to claim 89 wherein the pharmaceutical formulation comprises a propellant and wherein the administration comprises aerosolizing the antifungal agent by opening a valve to release the pharmaceutical formulation.

96. A method according to claim 89 wherein the pharmaceutical formulation is a liquid and wherein the administration comprises aerosolizing the liquid using a compressed gas and/or a vibrating member.

98. A unit dose receptacle comprising an aerosolizable pharmaceutical formulation for delivering from 5 mg to 10 mg of amphotericin B when aerosolized.
